(19) United States
(12) Patent Application Publication
(10) Pub. No.: US 2014/0369933 A1
(43) Pub. Date: Dec. 18, 2014

(54) FLUORINE-18 LABELED HYDROGEN ION PROBES

(71) Applicant: The Trustees of The University of Pennsylvania, Philadelphia, PA (US)

(72) Inventors: Alexander Victor Kachur, Media, PA (US); Anatoliy V. Popov, Philadelphia, PA (US); Edward James Delikatny, Havertown, PA (US); Joel S. Karp, Glenside, PA (US)

(21) Appl. No.: 14/352,134

(22) PCT Filed: Oct. 9, 2012
(86) PCT No.: PCT/US2012/059322

§ 371 Date: Apr. 16, 2014

Related U.S. Application Data
(60) Provisional application No. 61/550,644, filed on Oct. 24, 2011.

(51) Int. Cl. A61K 51/04; C07B 59/00

(52) U.S. Cl. CPC A61K 51/04; C07B 59/001
USPC 424/1.89; 558/58

(57) ABSTRACT

Fluorine-containing molecules include fluorine-18 labeled hydrogen ion indicator molecules and methods of making and using the same. Fluorine atoms incorporated into the indicator molecules provides novel structural modifications to the precursor molecules, shifting the absorbance maxima relative to their non-fluorinated precursor/cogeners. These molecules are useful for non-invasive in vivo measurement of blood volume, blood flow and pH in biological objects. Compounds are functionalized by a radioisotopically enriched fluorine-18 label and indicator molecule with structure derived from triarylmethane derived indicators, such as phenol red, cresol red, cresol purple, thymol blue, bromophenol red, naphthol blue, phenolphthalein, cresolphthalein, thymolphthalein, naphtholphthalein, gentian violet (methyl violet 10B), methyl violet 2B, methyl violet 6B, leucomalachite green (malachite green), brilliant green, pararosaniline, fuchsines, or salts thereof. During its radioactive decay, fluorine-18 produces high-energy positrons, which can be detected by positron emission tomography (PET) or single photon emission computer tomography (SPECT).

### CROSS REFERENCE TO RELATED APPLICATIONS

The present application claims priority to U.S. Patent Application No. 61/550,644, filed Oct. 24, 2011, which is incorporated by reference herein in its entirety.

### TECHNICAL FIELD

The present invention describes novel fluorine-containing, including fluorine-18 labeled, hydrogen ion indicator molecules and methods of making and using the same.

### BACKGROUND

Contemporary medical imaging depends largely upon the use of radioisotopes. In particular, Positron Emission Tomography (PET) and Single Photon Emission Computer Spectroscopy (SPECT) imaging are useful tools for diagnosing and monitoring a range of physiological conditions. Fluorine-18 is one of the most useful positron emitting radionuclides currently being used in clinical nuclear medicine diagnosis. Compounds incorporating this radioisotope produce superior high-resolution images and quantitative regional uptake of tissues. The 110-min half-life of fluorine-18 allows production and distribution of radioisotopically-labeled molecular fluorine to nuclear medicine facilities near a cyclotron center. The relatively long physical half-life of fluorine-18 also permits PET studies of moderately slow physiological process. Decay of fluorine-18 is largely by positron emission (97%), and the emitted positron is of relatively low energy (maximum 0.635 meV) and thus has a short mean range (2.39 nm in water). Fluorine-18 is readily available from both particle accelerators and nuclear reactors using a wide variety of nuclear reactions, and can be produced at specific activities approaching the theoretical limit of 1.17×10⁹ ci/mmol.

Despite the utility of fluorine-18, there are only a very small number of methods to introduce fluorine-18 into organic molecules. Direct electrophilic fluorination is rarely used as a synthetic method for labeling of complex organic molecules with the positron emitting isotope ¹⁸F, because this reaction usually results in non-specific decomposition of the reagent.

There is an urgent need for the development of new methods for incorporating fluorine, including fluorine enriched in ¹⁸F, into organic molecules, as well as the fluorinated agents that result from such methods.

### SUMMARY

(see attached)

### BRIEF DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific methods, devices, and systems disclosed. In addition, the drawings are not necessarily drawn to scale. In the drawings:

FIG. 1 shows a typical chromatogram of semi-preparative HPLC separation of the product of Cresol Purple fluorination, as described in Example 1 (abscissa in minutes). The bottom part represents the UV absorption, which cannot be resolved due to high extinction coefficients of the triarymehane indicators. The top curve (radioactivity) shows presence of two separable monofluorinated isomers (MFCP, 20 minutes and 21.5 minutes), difluorinated DFCP at 23 minutes, trifluorinated TFCP at 26 minutes, and small amount of tetrafluorinated QFCP at 29 minutes.

FIG. 2 shows analytical HPLC signals of the products of Phenol Red fluorination separated by HPLC. The left panel shows a diagram of the fraction containing MFPR and the right panel shows mostly DFPR. Signals from the product prepared by the Friedel-Crafts reaction were identical.

FIG. 3 shows the pH dependence of A560 for Phenol Red, A564 for MFPR, and A570 for DFPR, demonstrating the shift in pKa due to the introduction of fluorine atoms into the molecule.

FIG. 4 is a graphical representation of the absorption maxima of the basic forms, pKₐ values and color change range of the several fluorinated phenolsulfonphthalein derivatives described in the Examples, showing a potential range of application of pH indicators.

FIG. 5 shows the chromatographs of analytical HPLC of three fractions, containing fluorinated derivatives of Cresol Purple (see Example 3). The precursor (CP) is represented as a single peak on the upper curve, while other maxima are splitting into several closely located peaks, corresponding to different structural isomers of the products.

FIG. 6 represents the main structural isomers of MFCP, DFCP, and TFCP, shown for the most symmetrical acidic zwitterion form. The upper left formula reflects a numbering of carbon atoms in phenol rings used in text.

FIG. 7 shows the relationship between pH and relative absorption maxima for Cresol Purple derivatives, demonstrating increasing wavelength and decreasing pKₐ as result of successive addition of fluorine atoms.

FIG. 8 shows the dependence of radiochemical yields of the fluorinated derivatives of Cresol Purple on the ratio of amount of precursor to fluorine, as described in Example 4.

### DETAILED DESCRIPTION OF ILLUSTRATIVE EMBODIMENTS

The present invention describes novel fluorine-containing, including fluorine-18 labeled, hydrogen ion indicator molecules and methods of making and using the same. In many cases, the incorporation of fluorine atoms (even non-radiolabeled fluorine) into the indicator molecules provides novel structural modifications to the precursor molecules, shifting the absorbance maxima relative to their non-fluorinated precursor/cogeners. These molecules are useful for non-invasive in vivo measurement of blood volume, blood flow and pH in various (including biological) objects. In certain embodiments, the compounds two types of functionalization: a radioisotopically enriched fluorine-18 label and indicator molecule with structure derived from triarylmethane derived indicators, such as phenol red, cresol red, cresol purple, thymol blue, bromophenol red, naphthol blue, phenolphthalein, cresolphthalein, thymolphthalein, naphtholphthalein, gentian violet (methyl violet 10B), methyl violet 2B, methyl violet 6B, leucomalachite green (malachite green), brilliant green, pararosaniline, fuchsines, or salts thereof. During its radioactive decay, fluorine-18 produces high-energy positrons, which can be detected by positron emission tomography (PET) or single photon emission computer tomography (SPECT). Indicator portion of the molecule absorbs light or in pH-dependent manner. Combination of radiation and optical analysis provides a new method for non-invasive in vivo pH measurement in biological objects.

The present invention may be understood more readily by reference to the following description taken in connection with the accompanying Figures and Examples, all of which form a part of this disclosure. It is to be understood that this invention is not limited to the specific products, methods, conditions or parameters described and/or shown herein, and that the terminology used herein is for the purpose of describing particular embodiments by way of example only and is not intended to be limiting of any claimed invention. Similarly, unless specifically otherwise stated, any description as to a possible mechanism or mode of action or reason for improvement is meant to be illustrative only, and the invention herein is not to be constrained by the correctness or incorrectness of any such suggested mechanism or mode of action or reason for improvement. Throughout this text, it is recognized that the descriptions refer both to the features of certain chemicals or chemical compositions, as well as the methods of making and using these chemicals or chemical compositions, and vice versa.

In the present disclosure the singular forms “a,” “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about.” In other cases, the gradations used in a series of values may be used to determine the intended range available to the term “about” for each value. Where present, all ranges are inclusive and combinable. That is, references to values stated in ranges include every value within that range.

It is to be appreciated that certain features of the invention which are, for clarity, described herein in the context of separate embodiments, may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are described herein.

Whenever a group of this invention is described as being “optionally substituted” that group may be unsubstituted or substituted with one or more of the substituents described for that group. Likewise, when a group is described as being “unsubstituted or substituted,” if substituted, the substituent may be selected from the same group of substituents. Unless otherwise indicated, when a substituent is deemed to be “optionally substituted,” or “substituted” it is meant that the substituent is a group that may be substituted with one or more group(s) individually and independently selected.

Each of the following terms (e.g., “alkyl,” “heteroalkyl,” “alkoxy,” “aryl,” and “heteroaryl”) include both substituted and unsubstituted forms of the indicated group, unless indicated otherwise.

The term “alkyl,” by itself or as part of another substituent, means, unless otherwise stated, a straight or branched chain, or cyclic hydrocarbon group (cycloalkyl), or combination thereof, which may be fully saturated, mono- or polyunsaturated and can include di- and multivalent radicals, and can have a number of carbon atoms optionally designated (e.g., C₁₋₃ means one to three carbons). Examples of saturated hydrocarbon groups include, but are not limited to, groups such as methyl, ethyl, n-propyl, and isopropyl. C₁₋₃ alkyls are preferred.

An alkyl group of this invention may be substituted or unsubstituted. When substituted, the substituent group(s) may be one or more group(s) independently selected from cycloalkyl, aryl, heteroaryl, heteroalicyclyl, hydroxy, alkoxy, aryloxy, mercapto, alkylthio, arylthio, cyano, halo, oxo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, amino or substituted amino, protected hydroxyl, protected amino, protected carboxy and protected amido groups.

The term “alkoxy” is used in its conventional sense, and refers to those alkyl groups attached to the remainder of the molecule via an oxygen atom. Alkoxy groups include, but are not limited to methoxy, ethoxy, propoxy, trifluoromethoxy and difluoromethoxy.

The term “aryl” refers to a chemical moiety comprising at least one cyclic aromatic carbocycle, preferably having 4, 6, 8, or 10 carbon atoms. Exemplary aryl groups include, but are not limited to, phenyl and naphthyl. The term “heteroaryl” refers to a chemical moiety comprising at least one cyclic aromatic group containing at least one heteroatom (N, O, S). Exemplary heteroaryl groups include, but are not limited to pyridyl, furyl, thienyl, pyrrolyl, oxazolyl, imidazolyl, pyrazolyl, and triazolyl.

The terms “halo” or “halogen,” by themselves or as part of another substituent, mean, unless otherwise stated, a fluorine, chlorine, bromine, or iodine atom.

The term “heteroalkyl,” by itself or in combination with another term, means, unless otherwise stated, a stable straight or branched chain, or cyclic hydrocarbon group, or combinations thereof, consisting of a number of carbon atoms and at least one heteroatom selected from the group consisting of O, N, Si and S, and wherein the nitrogen, carbon and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃.

As used herein in the context of organic compounds, the incorporated fluorine may be present either in its natural abundance of isotopes or statistically enriched in the radioisotope ¹⁸F. Unless otherwise specified, an organic chemical structure shown as having an “F” moiety attached at a given position, is generally intended to refer to separate embodiments in which the fluorine moiety is present either having its natural abundance of isotopes or being statistically enriched in the radioisotope ¹⁸F. Where an organic chemical structure is shown as having an “¹⁸F” moiety attached at a given position, that moiety is intended to reflect a statistical enrichment of this radioisotope at that position (while possible that an individual molecule may contain an ¹⁸F atom at this position, the descriptions of structures as used herein are intended to reflect compounds comprising a plurality of structurally consistent molecules). Where enriched in the radioactive ¹⁸F isotope, this isotope is prepared artificially.

The invention describes at least four groups of novel compounds which include the products of the reactions described herein. They are mono-, di-, tri-, and tetrafluorinated derivatives of the precursor molecule with general formulas:

Phenolsulfonphthalein:

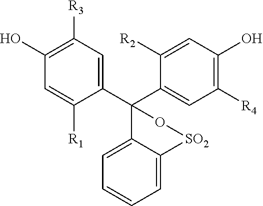

Phenolphthalein:

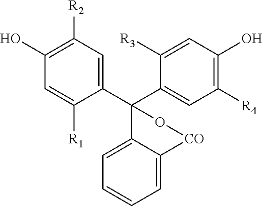

Naphtholsulfonphthalein:

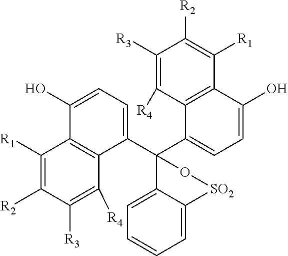

Naphtholphthalein:

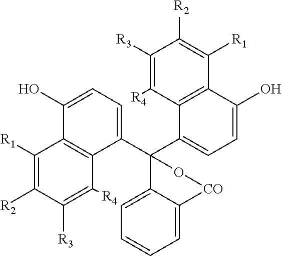

where R₁, R₂, R₃, and R₄ are defined below in various embodiments.

The distinctive features of these compounds are having: three aromatic rings attached to central carbon atom with one ring having sulfonic or carboxyl group in ortho-position (sulfonic/carbonic ring) and two rings with hydroxyl group in para-position (phenol/naphthol rings) to central atom; the fluorine atoms attached in: meta-positions (regarding to the central carbon atom) of one and/or both phenol/naphthol rings. In various embodiments, the fluorine may be statistically present in their natural abundance of isotopes. In other embodiments, the fluorine is statistically enriched in the radioisotope ¹⁸F, above its natural isotopic abundance relative to the ¹⁹F isotope.

Embodiments of the present invention include those compounds having a structure of Formula I, II, III, or IV or a salt thereof:

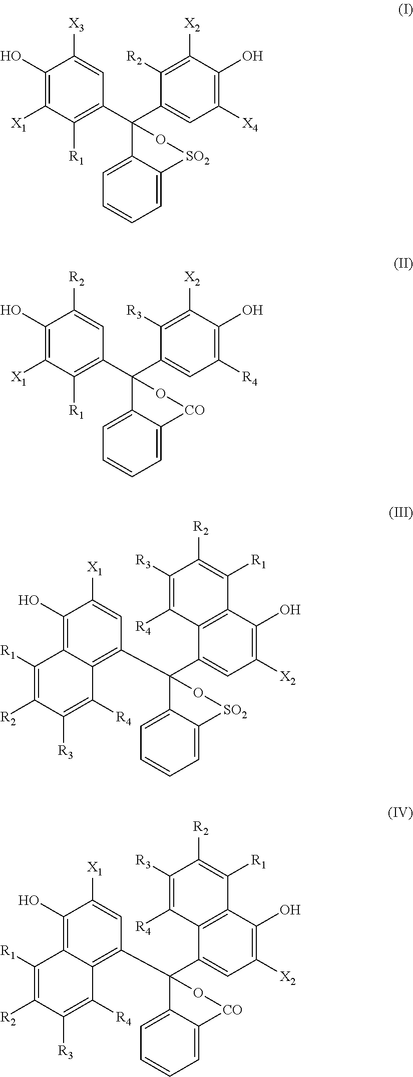

wherein

X₁ and X₂ are independently, at each occurrence, hydrogen or fluorine;

R₁, R₂, R₃, and R₄, are independently, at each occurrence, hydrogen, optionally substituted C₁₋₆ alkyl; optionally substituted C₃₋₆ cycloalkyl, optionally substituted C₂₋₆ alkenyl; optionally substituted alkoxy, optionally substituted aryl, optionally substituted heteroalkyl, optionally substituted heteroaryl, or halo;

X₃ and X₄ are independently, at each occurrence, hydrogen, fluorine, chlorine, bromine, iodine, optionally substituted C₁₋₆ alkyl, optionally substituted C₃₋₆ cycloalkyl, optionally substituted C₂₋₆ alkenyl; optionally substituted alkoxy, optionally substituted aryl, optionally substituted heteroalkyl, or optionally substituted heteroaryl;

provided at least one of X₁, X₂, X₃, and X₄ is fluorine;

in each case the fluorine being present either at its natural abundance of isotopes at each occurrence or statistically enriched in the radioisotope ¹⁸F above its natural isotopic abundance relative to the ¹⁹F isotope at each occurrence. In other independent embodiments, where present, the fluorine is statistically present at its natural abundance of isotopes. In other embodiments, where present, the fluorine is statistically enriched in the radioisotope ¹⁸F above its natural isotopic abundance relative to the ¹⁹F isotope.

Other embodiments provide these compounds wherein R₁, R₂, R₃, and R₄ are independently, at each occurrence, hydrogen or linear or branched C₁₋₃ alkyl, preferably methyl or isopropyl.

Still other embodiments provide these compounds wherein either (a) X₁=fluorine, X₂=X₃=X₄=H; or (b) X₁=X₂=fluorine, X₃=X₄=H; or (c) X₁=X₂=X₃=fluorine, X₄=H, or (d) X₁=X₂=X₃=X₄=fluorine. Consistent with the above descriptions, the fluorine may be statistically present either at its natural abundance of isotopes or enriched in the radioisotope ¹⁸F above its natural isotopic abundance relative to the ¹⁹F isotope.

Still other independent embodiments provide compounds having a structure described as:

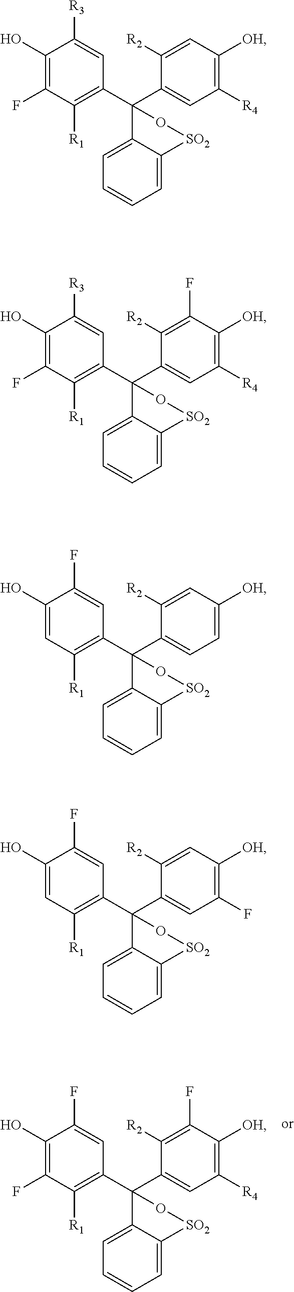

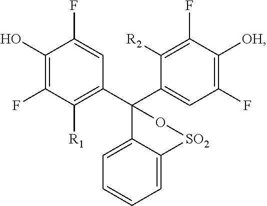

where R₁, R₂, R₃, and R₄, are independently, at each occurrence, hydrogen, optionally substituted C₁₋₆ alkyl (preferably methyl or isopropyl); optionally substituted C₃₋₆ cycloalkyl, optionally substituted C₂₋₆ alkenyl; optionally substituted alkoxy, optionally substituted aryl, optionally substituted heteroalkyl, optionally substituted heteroaryl, or halo; and

where F represents either fluorine present at its natural abundance of isotopes at each occurrence or fluorine statistically enriched in the radioisotope ¹⁸F above its natural isotopic abundance relative to the ¹⁹F isotope at each occurrence. In certain of these embodiments, R₁, R₂, R₃, and R₄ are independently hydrogen, methyl, or isopropyl.

Still other embodiments provide compounds having a structure described as:

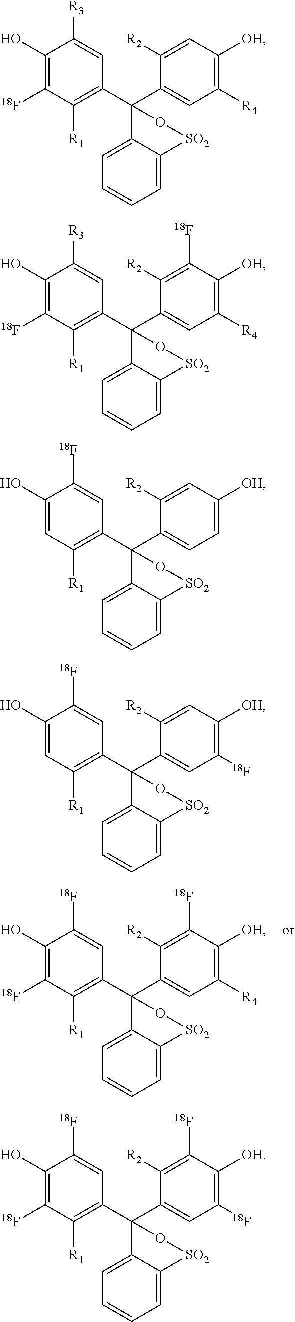

where R₁, R₂, R₃, and R₄, are independently, at each occurrence, hydrogen, optionally substituted C₁₋₆ alkyl (preferably methyl or isopropyl); optionally substituted C₃₋₆ cycloalkyl, optionally substituted C₂₋₆ alkenyl; optionally substituted alkoxy, optionally substituted aryl, optionally substituted heteroalkyl, optionally substituted heteroaryl, or halo; and

where ¹⁸F represents fluorine that is statistically enriched in the radioisotope ¹⁸F above its natural isotopic abundance relative to the ¹⁹F isotope. In certain of these embodiments, R₁, R₂, R₃, and R₄ are independently hydrogen, methyl, or isopropyl.

In certain embodiments, the compounds have a structure according to:

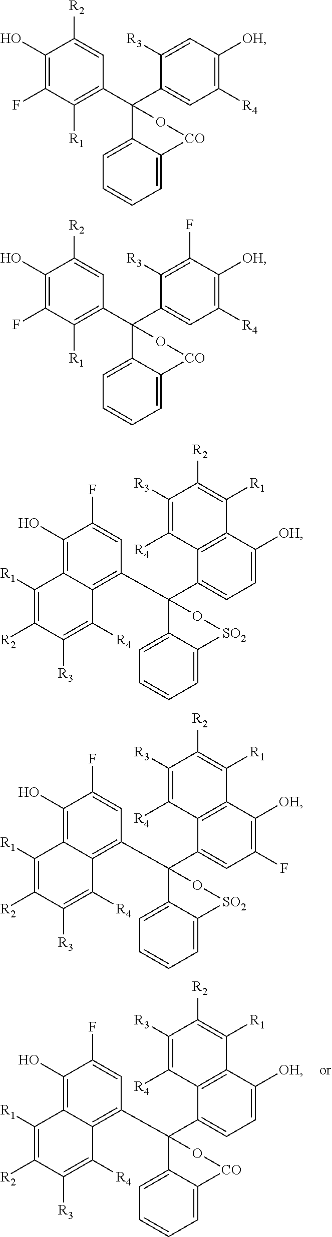

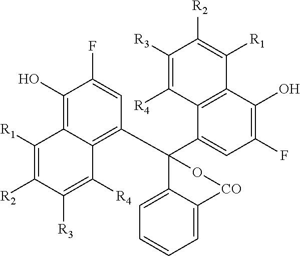

where R₁, R₂, R₃, and R₄, are independently, at each occurrence, hydrogen, optionally substituted C₁₋₆ alkyl (preferably methyl or isopropyl); optionally substituted C₃₋₆ cycloalkyl, optionally substituted C₂₋₆ alkenyl; optionally substituted alkoxy, optionally substituted aryl, optionally substituted heteroalkyl, optionally substituted heteroaryl, or halo; and

where F represent either fluorine being present at its natural abundance of isotopes or fluorine that is statistically enriched in the radioisotope ¹⁸F above its natural abundance relative to the ¹⁹F isotope. In certain of these embodiments, R₁, R₂, R₃, and R₄ are independently hydrogen, methyl, or isopropyl, and preferably all hydrogen.

In other embodiments, the compounds have a structure according to:

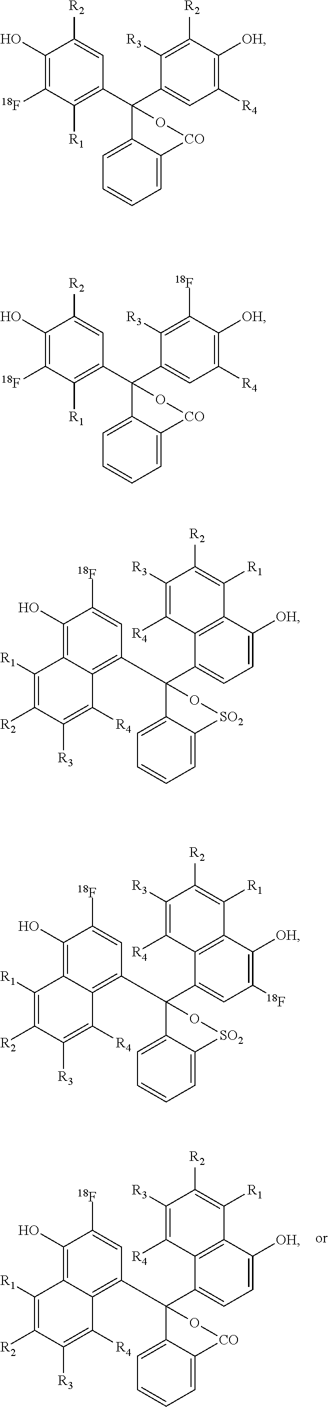

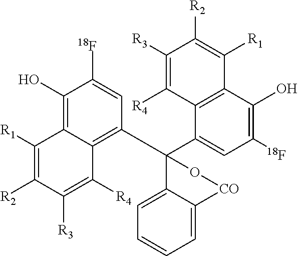

where R₁, R₂, R₃, and R₄, are independently, at each occurrence, hydrogen, optionally substituted C₁₋₆ alkyl (preferably methyl or isopropyl); optionally substituted C₃₋₆ cycloalkyl, optionally substituted C₂₋₆ alkenyl; optionally substituted alkoxy, optionally substituted aryl, optionally substituted heteroalkyl, optionally substituted heteroaryl, or halo; and

where ¹⁸F represents fluorine that is statistically enriched in the radioisotope ¹⁸F above its natural abundance relative to the ¹⁹F isotope. In certain of these embodiments, R₁, R₂, R₃, and R₄ are independently hydrogen, methyl, or isopropyl, or alternatively all hydrogen.

The present disclosure also describes and claims new methods of providing novel compounds. See Examples below. Electrophilic fluorination of complex organic molecules with fluorine gas is not a straightforward process, significantly dissimilar to addition of other halogens to double bonds or substitution in aromatic cycles. Due to very high negative enthalpy of the reaction, fluorine gas is able to cause a cleavage of carbon-carbon bonds, which results in nonspecific fluorination and subsequent destruction of the target molecule. However, using the methods described herein, a series of triaryl-based indicators can and could be successfully fluorinated with molecular fluorine, even at ambient room temperature. Without intending to be bound by the correctness or incorrectness of any particular theory, it may be that the electrophilic fluorinations as described can be successfully carried out owing to the relatively low reactive aromatic protons, the positive resonance charge on the target part of molecules in acidic conditions through the use of carboxylic acid solvents, which may also act as a moderating agent converting fluorine into less aggressive acetyl hypofluorite, e.g. CH₃COOF.

Any statement that these reactions may be conducted at ambient temperatures does not mean that the reactions described herein cannot be conducted at lower or higher temperatures. In other systems with other unrelated molecules, experimentalists have been limited to using low temperature conditions—including performing electrophilic fluorinations with diluted fluorine gas at temperature down to as low as those of dry ice. To the extent that sufficient amounts of the precursors or their salts can be dissolved at such lower temperatures, similar or the same conditions may be used here. Using longer chain, perhaps perfluorinated, carboxylic acids as solvents or co-solvents may provide sufficient solubilities of the precursors to make these temperatures more viable.

Still further embodiments provide methods of preparing the compounds described above, including a compound of Formula I, II, III, or IV, or a salt thereof,

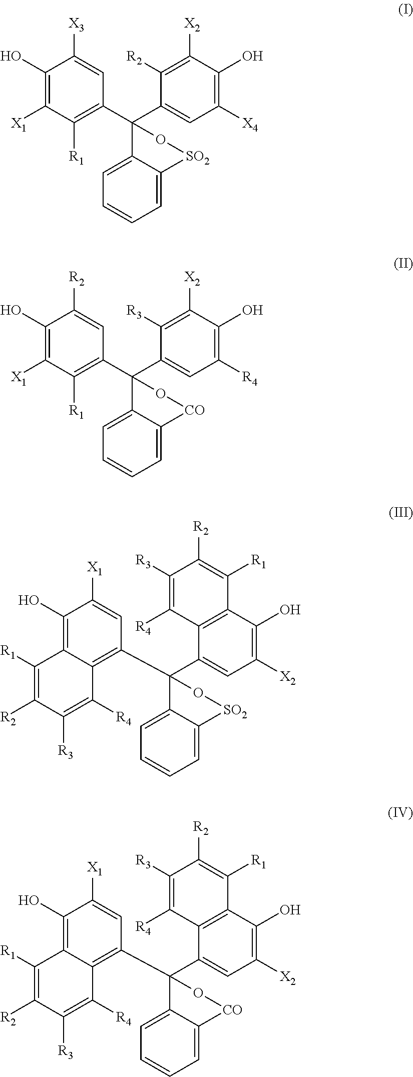

wherein

X₁ and X₂ are independently, at each occurrence, hydrogen or fluorine;

R₁, R₂, R₃, and R₄, are independently, at each occurrence, hydrogen, optionally substituted C₁₋₆ alkyl; optionally substituted C₃₋₆ cycloalkyl, optionally substituted C₂₋₆ alkenyl; optionally substituted alkoxy, optionally substituted aryl, optionally substituted heteroalkyl, optionally substituted heteroaryl, or halo;

X₃ and X₄ are independently, at each occurrence, hydrogen, fluorine, chlorine, bromine, iodine, optionally substituted C₁₋₆ alkyl; optionally substituted C₃₋₆ cycloalkyl, optionally substituted C₂₋₆ alkenyl; optionally substituted alkoxy, optionally substituted aryl, optionally substituted heteroalkyl, or optionally substituted heteroaryl; and

provided at least one of X₁, X₂, X₃, and X₄ is fluorine;

each method comprising reacting the corresponding precursor of Formula V-VIII, as appropriate for the particular compound of interest, with fluorine gas in a solvent:

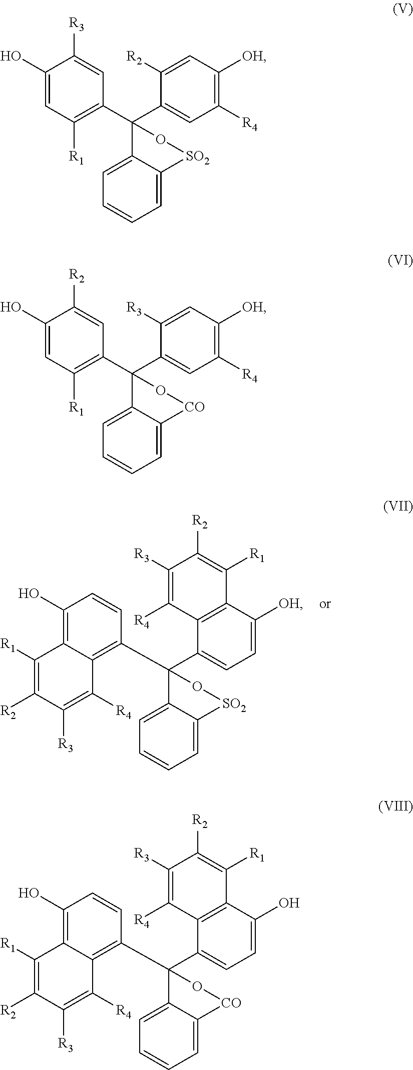

wherein

R₁, R₂, R₃, and R₄ are independently, at each occurrence, hydrogen, optionally substituted C₁₋₆ alkyl; optionally substituted C₃₋₆ cycloalkyl, optionally substituted C₂₋₆ alkenyl; optionally substituted alkoxy, optionally substituted aryl, optionally substituted heteroalkyl, optionally substituted heteroaryl, or halogen.

In various of these method embodiments, the fluorine gas is present as molecular fluorine comprising fluorine in its natural abundance of isotopes, and the at least one incorporated fluorine atom is statistically present at fluorine's natural abundance of isotopes. In other method embodiments, the fluorine gas is enriched in radiolabeled ¹⁸F isotope relative to the ¹⁹F isotope (generally present in a mixture of ¹⁸F₂, ¹⁸F¹⁹F, and ¹⁹F₂), and the at least one incorporated fluorine atom is incorporated statistically enriched in the radioisotope ¹⁸F above its natural isotopic abundance relative to the ¹⁹F isotope.

In these methods the compounds also include those wherein R₁, R₂, R₃, R₄, X₃ and X₄ are independently, at each occurrence, hydrogen or linear or branched C₁₋₃ alkyl, preferably methyl.

Other embodiments provide methods of preparing a pH indicator, each method comprising reacting a triarylmethane-based indicator precursor with fluorine gas (present either in its natural isotopic abundance or enriched in ¹⁸F) in a solvent, wherein the triarylmethane-based indicator precursor comprises phenol red, cresol red, cresol purple, thymol blue, bromophenol red, naphthol blue, phenolphthalein, cresolphthalein, thymolphthalein, naphtholphthalein, gentian violet (methyl violet 10B), methyl violet 2B, methyl violet 6B, leucomalachite green (malachite green), brilliant green, pararosaniline, fuchsines, or a salt thereof. These indicator precursors are known in the art. Additional embodiments include those wherein the triarylmethane-based indicator precursor is phenol red, cresol red, or cresol purple, thymol blue, phenolphthalein, naphtholphthalein, or a salt thereof.

The previously described methods also include those wherein the solvent comprises an optionally fluorinate or perfluorinated C₁₋₇ organic acid, for example CH₃COOH, CH₂FCOOH, CHF₂COOH, CF₃COOH, HCOOH, or a mixture thereof. In the specific cases of phenolphthalein, naphtholphthalein, and their derivatives, the preferred solvent is or comprises a fluorinated or perfluorinated organic acid, preferably CF₃COOH.

These methods also include those further comprising separating the products of the reaction by chromatography, preferably HPLC.

The invention also teaches embodiments including the compounds prepared by these methods. That is, certain embodiments provide the compounds prepared by the electrophilic fluorination of a precursor of Formula V, VI, VII, or VIII or a salt thereof:

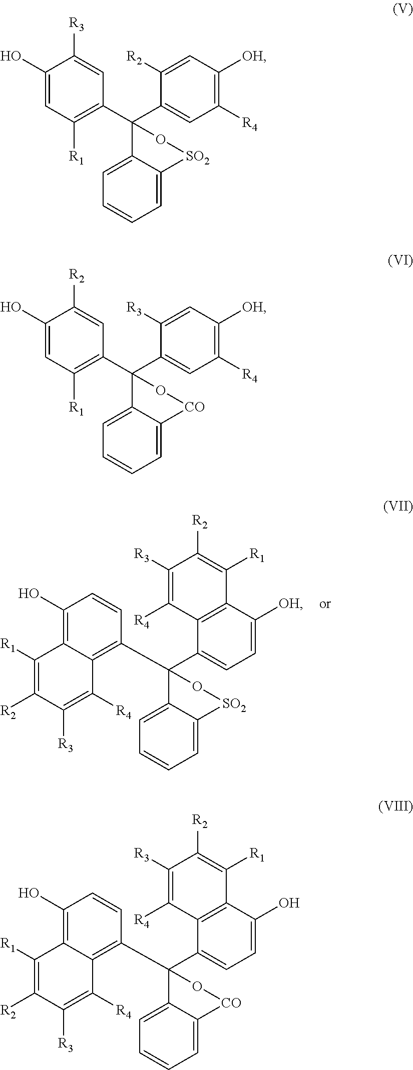

wherein

R₁, R₂, R₃, and R₄ are independently, at each occurrence, hydrogen, optionally substituted C₁₋₆ alkyl; optionally substituted C₃₋₆ cycloalkyl, optionally substituted C₂₋₆ alkenyl; optionally substituted alkoxy, optionally substituted aryl, optionally substituted heteroalkyl, optionally substituted heteroaryl, or halogen; and wherein

wherein the electrophilic fluorination reaction comprises reacting the precursor with molecular fluorine in a solvent, such that the compound incorporates and contains at least one fluorine atom per compound molecule. Independent embodiments provide that the compound molecule may contain one, two, three, four, or more fluorine atoms per compound molecule. In some embodiments, the molecular fluorine contains its natural abundance of fluorine isotopes (designated as ¹⁹F₂), and the at least one fluorine incorporated in the compound is present at its natural isotopic abundance. In other embodiments, the molecular fluorine is enriched in the radioisotope ¹⁸F above its natural isotopic abundance relative to the ¹⁹F isotope, and the at least one fluorine atom incorporated in the compound is enriched in the radioisotope ¹⁸F above its natural isotopic abundance relative to the ¹⁹F isotope. Where the fluorine gas is isotopically enriched with fluorine-18, it is generally presented as a statistical mixture of ¹⁹F, ¹⁸F¹⁹F, and ¹⁸F₂, the relative amounts of each depending on the total level of levels of ¹⁸F generated during its production (see Experimental).

It is also to be appreciated that each mono-, di-, tri, or tetra-fluoro compound which can be prepared from the reaction conditions being applied to the precursors described herein, even if not specifically mentioned (e.g., in addition to the specific structures described herein) or if also available by alternative synthetic means, is also considered a separate embodiment within the scope of this invention. See, for example, Example 3.

In certain embodiments, these compounds prepared by electrophilic fluorination are those wherein R₁, R₂, R₃, and R₄ are independently, at each occurrence, hydrogen or linear or branched C₁₋₃ alkyl, preferably methyl or isopropyl. In some embodiments, R₁, R₂, R₃, and R₄ are all hydrogen.

Other embodiments provide compounds prepared by the electrophilic fluorination of a triarylmethane dye precursor as described above, said precursor comprising phenol red, cresol red, cresol purple, thymol blue, bromophenol red, naphthol blue, phenolphthalein, cresolphthalein, thymolphthalein, naphtholphthalein, gentian violet (methyl violet 10B), methyl violet 2B, methyl violet 6B, leucomalachite green (malachite green), brilliant green, pararosaniline, fuchsines, or a salt thereof; wherein the electrophilic fluorination reaction comprises reacting the precursor with fluorine gas (radiolabeled or otherwise) in a solvent such that the compound contains at least one fluorine atom per compound molecule. Where the fluorine gas consists of fluorine gas at its natural isotopic abundance, the at least one incorporated fluorine atom will be statistically present at their natural isotopic abundance. Where the fluorine gas is enriched in levels of ¹⁸F, the at least one incorporated fluorine atom will be statistically enriched in the radioisotope ¹⁸F. Independent embodiments provide that the compound molecule may contain one, two, three, four, or more fluorine atoms per compound molecule.

Again, in some embodiments, the solvent is or comprises an organic acid, including CH₃COOH, CH₂FCOOH, CHF₂COOH, CF₃COOH, HCOOH, or a mixture thereof and/or a fluorinated or perfluorinated C₃₋₇ organic acid. In the specific cases of phenolphthalein, naphtholphthalein, and their derivatives, the preferred solvent is or comprises a fluorinated or perfluorinated organic acid, preferably CF₃COOH

These compounds may also be prepared by methods further comprising separating the products of the reaction by chromatography, especially HPLC or semi-preparative HPLC.

In various embodiments of the methods and compounds derived therefrom, the precursor may be introduced into the solvent in the form of salt with one or more of any metal cation (e.g., mono- or disodium salt), which enables its solubility in reaction solvent.

In various embodiments of the methods and compounds derived therefrom, the fluorine gas (radiolabeled or not) is preferably diluted by an inert gas, such as He, Ne, Ar, Kr, Xe to low (below about 10 wt %) concentration. The reaction is preferably performed by bubbling of the fluorine gas through solution of the precursor in the solvent at room or lowered temperature.

While not intending to be bound by any particular theory or mechanism, the overall reaction processes (shown here using radiolabeled fluorine) are believed to be described by the following equations:

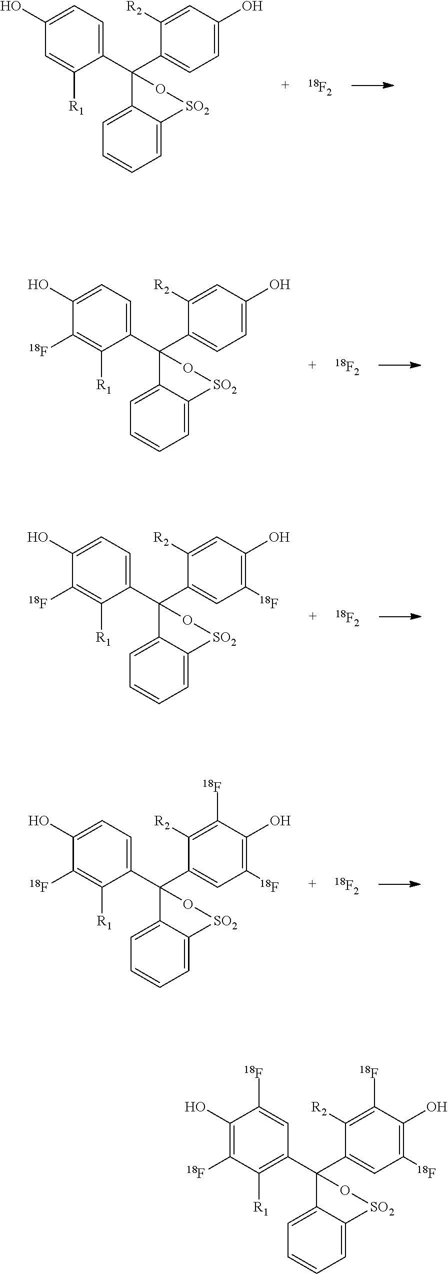

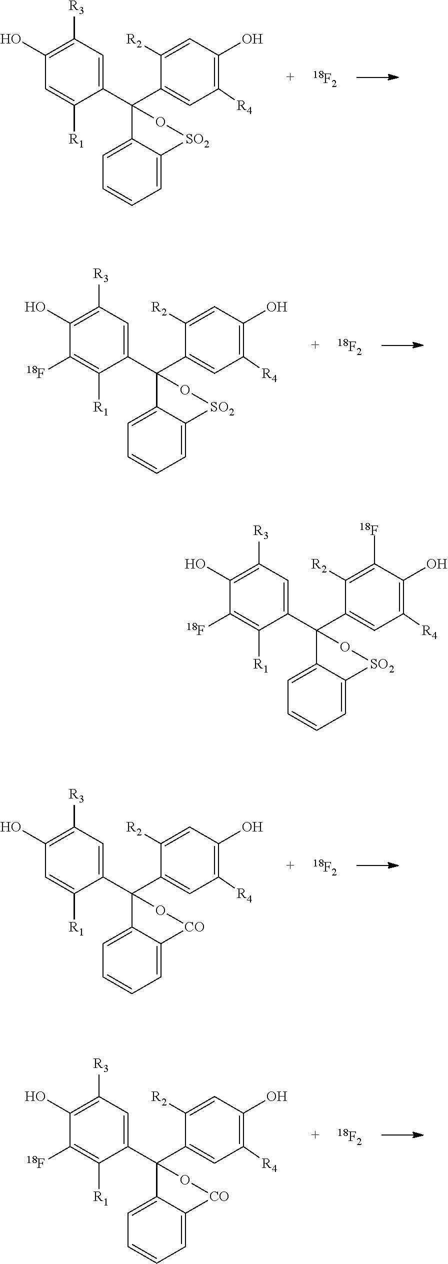

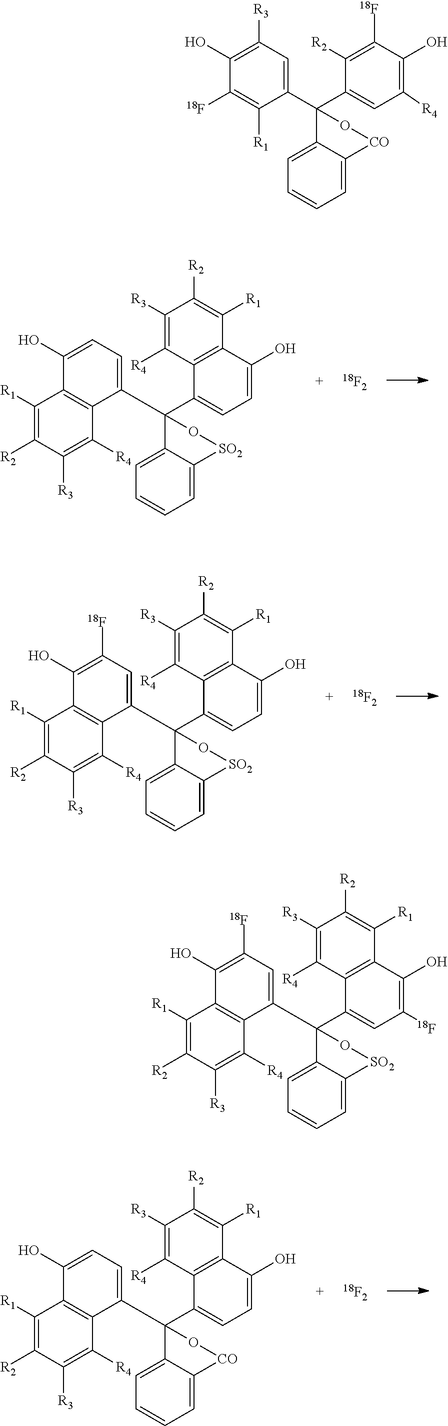

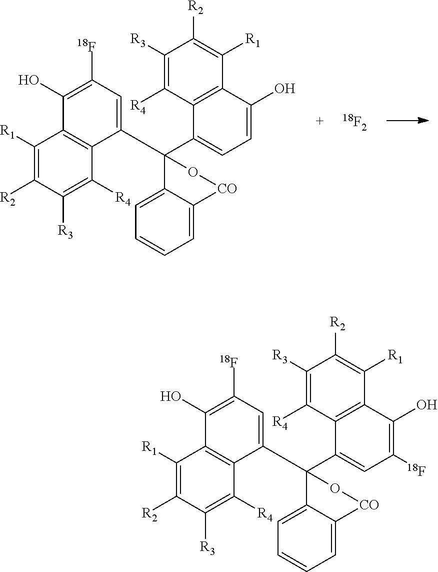

To this point, the invention has been described in terms of compounds and methods of making the same, but the invention also includes methods of using these compounds.

For example, some embodiments provide methods of determining the concentration of any of the preceding compounds in a biological object, for example blood, tumor or kidney, using positron emission tomography (PET) or single photon emission computer spectroscopy to determine compound concentration, blood volume, blood flow through the organ or tissue, or kidney activity.

Other embodiments provide methods of measuring the in vivo pH or pH changes (such as associated with hypoxia or diabetes) in a biological object, for example a tumor or blood, each method comprising incorporating the at least one of any of the previously described compounds in said biological object and measuring a spectral response of the compound using light in the visible or ultraviolet ranges, said spectrum representative of the pH of the biological object. A method of detecting blood flow and/or pH changes associated with hypoxia or diabetes in a patient, said method comprising one or more of the following steps: administering to the patient at least one of the previously described compounds; optionally allowing the compound to distribute within a biological object (for example blood or a tumor) within the patient; identifying the biological object within the patient containing the compound, and measuring a spectral response of the compound using light in the visible or ultraviolet ranges, said spectral response being representative of the internal pH of the patient in combination with determining the concentration of any of the preceding compounds in a biological object using positron emission tomography (PET) or single photon emission computer spectroscopy.

The compounds described herein can also be used for 3D mapping of hydrogen ion concentration in the object by the combination of optical absorption by basic form of the indicator with PET imaging, which detects distribution of the probe in the object. Until recently, phenol red was used for the investigation of blood flow through the kidney by intravenous drug injection and observing its renal excretion. This method is becoming obsolete; however, labeling of phenol red and its derivatives with PET isotope could result in renewed interest in this technique. The covalent radii of hydrogen and fluorine are quite close, so replacement of hydrogen atom by fluorine is not expected to cause a drastic change of its structure and biological behavior of the molecule. Subsequently, pharmacokinetics of ¹⁸F labeled Phenol Red derivatives should be similar to parental compound: rapid (within minutes) biodistribution and fast (with half-life 20-30 minutes) mostly renal clearance. Since the PETt technique allows direct real time in vivo observation of the positron emitter, ¹⁸F labeled Phenol Red derivatives will allow observation of a blood volume in the tissues and organs as well as following of the activity of the kidney in space- and time-resolved manner. Phenol Red derivatives synthesized in the current paper have aliphatic substituting groups and therefore they are more hydrophobic. Most likely these derivatives will have slightly altered pharmacokinetic properties with clearance shifted to hepatobilliary system and increased half-life in the organism. This suggests more possibilities for in vivo application of these ¹⁸F-labeled derivatives.

¹⁸F-labeled pH indicators represent a new type of probe for in vivo investigation of hydrogen ion concentration. These compounds allow for two modes of detection: pH-dependent optical absorption and concentration-dependent gamma-radiation by PET technique.

Absorption of light by acidic (HA) and basic (A⁻) forms of the indicator molecule allows precise determination of the pH by the Henderson-Hasselbalch equation:

pH=pKₐ+log [A⁻]/[HA].

Determination of the concentration of the basic forms [A⁻] for triarylmethane indicators is relatively easy due to their high absorption wavelengths. However, acidic forms [HA] cannot be reliably detected in biological objects because they absorb in UV range (300-450 nm), interfering with other biomolecules and Rayleigh scattering from cells and organelles. This problem can be overcome by determination of the total amount of the probe in the object by another (non-optical) method. Labeling of indicator molecule with ¹⁸F provides an opportunity to apply PET technique for determination of the total indicator concentration [A]ₜₒₜₐₗ and calculation of the pH by the formula:

pH=pKₐ+log [A⁻]/([A]ₜₒₜₐₗ−[A⁻]).

Combining the 3-dimensional distribution of basic form of indicator with the PET image of its concentration will lead to in vivo 3D mapping of the pH in the biological object.

A functional probe useful for this kind of an investigation should contain a radioactive PET label (¹⁸F) within the indicator molecule with optimal value of pKₐ (between 6 and 8), and appropriate absorption maximum of basic form. Application of these indicators for mammal objects must also consider the presence of hemoglobin, which has strong absorption peaks for both Hb (555 nm) and HbO₂ (542 nm and 577 nm) and could interfere with indicator. The following radiolabeled compounds described herein would appear to be particularly attractive for in vivo applications: monofluorocresolsulfonphthalein (MFCP); difluorocresolsulfonphthalein (DFCP); trifluorocresolsulfonphthalein (TFCP); difluorothymolsulfonphthalein (DFTB); monofluoronaphtholphthalein (MFNP); and difluoronaphtholphthalein (DFNP),

### EXAMPLES

The following examples are not intended to limit the scope of the described invention, and the reader should not interpret them in this way. However, each of the compounds and specific experimental parameter or parameters is considered to be individual combinable embodiments of the present invention. Moreover, while the procedures and descriptions are given in terms of fluorine enriched in ¹⁸F isotopes, it should be appreciated that the same procedures may be conducted with non-enriched molecular fluorine (i.e., fluorine in its natural isotopic abundance), producing analogous fluorinated derivatives containing non-enriched fluorine atoms, and that such procedures and resulting products are also deemed to be within the scope of the present invention.

### Example 1

### Materials and General Methods

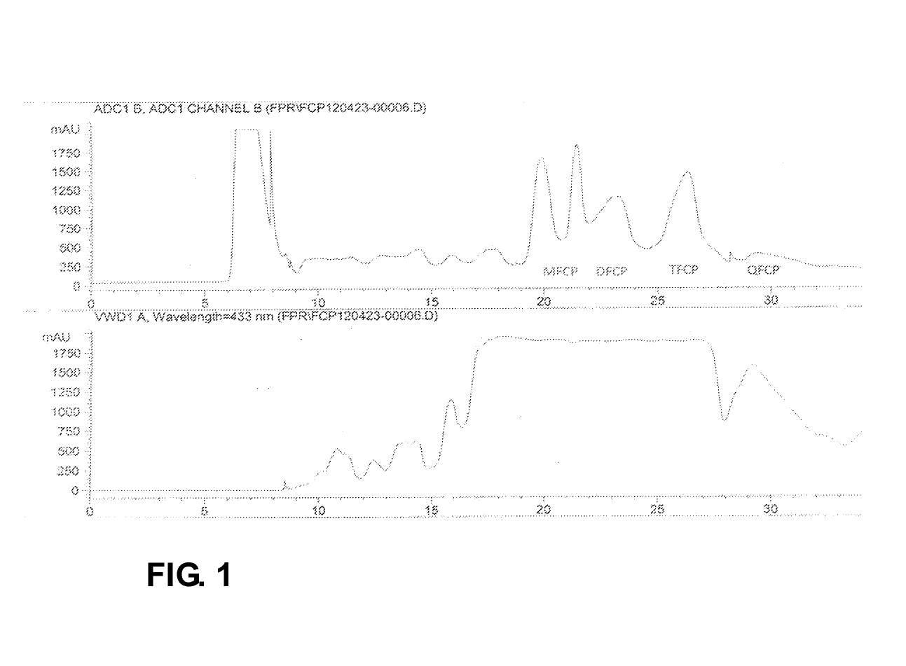

All reagents, including indicators, were purchased from Sigma-Aldrich. Labeling of the molecules with a statistical enrichment of ¹⁸F was performed at Cyclotron Facility in the Department of Radiology at the University of Pennsylvania using a custom-made electrophilic fluorination unit. [¹⁸F]—F₂ gas (i.e., flourine molecules enriched in ¹⁸F) was prepared by the reaction ²° Ne(d,a)¹⁸F using an IBA 18/9 Cyclone accelerator. The labeling reaction was performed by bubbling the 0.1% [¹⁸F]—F₂ in Ne (a total 100-200 mCi of activity in 35 micromoles of ¹⁹F carrier) for 5 minutes through freshly prepared solution of the sodium or disodium salt of correspondent indicator in glacial acetic acid (2 mg/mL). The reaction mixture was then evaporated under vacuum at 120° C., re-dissolved in 2 mL of water, injected into a semi-preparative HPLC column (Phenomenex, Synergi 4 μm Hydro-RP 80 Å 10×250 mm) and eluted by 25% ethanol-water or acetonitrile-water buffer at 2 mL/min rate with simultaneous detection of radioactivity and absorption at 430 nm. Under these conditions elution times for the products of interest were approximately 15 min for Phenol Red, and from 20 to 30 min for products of its fluorination. Phenol Red derivatives with a methyl group in the molecule have increased hydrophobic character, which resulted in higher retention times for both the precursors and the products of their fluorination. An example of semi-preparative HPLC separation of the products is shown on FIG. 1.

Separations of the individual compounds were performed mostly on the basis of the radioactivity peaks, because the UV signals from semi-prep HPLC were not resolved due to the very high extinction coefficients of the indicators. In certain cases, consecutive fractions (typically, but not limited to 4 mL) were collected and used for further analysis. In order to analyze the product mixtures, fractions were collected and analyzed by analytical HPLC, mass-spectroscopy, and UV-VIS spectroscopy. In the case of the products of Cresol Purple, proton NMR was also used. Presence of individual components in the fractions was confirmed by analytical HPLC performed with a 4.6×250 mm column of the same type and gradient ethanol(acetonitrile)-ammonium acetate (0.1 M, pH=4.7) buffer. Incorporation of the fluorine atoms into the indicator molecule was determined by the determination of the molecular mass of the compounds using a mass-spectroscopy on Thermo Fisher Scientific (Bremen, Germany) Orbitrap Exactive. The molecular mass of the products was determined after complete decay of the radioactive label, because relative amount of fluorine-18 in radioactive [¹⁸F]—F₂ gas was very small and would not give a distinct signal. The values of pKa of the indicators were determined by UV-VIS spectroscopy from the pH dependence of absorption of their basic forms and other spectroscopic investigations of the products were performed by Beckman-Coulter DU 530 spectrophotometer using acetate, phosphate, TRIZMA, and borate buffers for different values of pH. The number of fluorine atoms in the molecule was also determined by comparison of the ratio of sample activity to absorption of the basic forms of indicators. For the samples from the same preparation it was equal to a set of increasing simple numbers, corresponding to successive introduction of fluorine atoms into the molecule determined by mass-spectroscopy.

Molecular mass of the compounds was determined by mass-spectroscopy using a Thermo Fisher Scientific (Bremen, Germany) Orbitrap Exactive. Electrospray Ionization (ESI) was performed, introducing the sample into the instrument at a flow rate of 5 μL/min. The experiment was done in both positive and negative modes with a capillary temperature of 275° C. In positive-ion mode, the electrospray voltage was set to 4.2 kV, the capillary voltage to 50 V, and the tube lens offset to 145 V. The nitrogen sheath gas flow was optimized at 10 arbitrary units.

In addition, Solvent-Assisted Inlet Ionization (SAII) on the Orbitrap Exactive was used to verify the ESI results in both positive and negative mode. SAII is a novel ionization technique, in which the ion source was removed, a fused silica capillary tube without the coating is placed in the inlet of the mass spectrometer and the sample is pulled by vacuum through the fused silica tube into a heater orifice into the mass spectrometer. This was performed in both positive and negative mode with a capillary temperature of 325° C., capillary voltage ±90 V and tube lens voltage ±120 V. The data was controlled and analyzed using Xcalibur software (Thermo Fisher Scientific) for both ionization techniques.

In the case of Cresol Purple, additional analyses were conducted. In order to characterize individual fluorinated derivatives of Cresol Purple, correspondent fractions were collected from five preparations and repeatedly purified by semi-preparative HPLC. Further characterization was conducted using proton NMR, performed on a Bruker DMX-360 spectrometer using deuterated chloroform/methanol solvent. It was not possible to obtain clear ¹⁹F spectra of the products due to small amounts of the compounds and existence of different structural isomers and acid-base forms.

Independent syntheses of the reaction products of the fluorination of Phenol Red were performed using nonradiolabeled precursors by a Friedel-Crafts reaction between 2-sulfobenzoic acid cyclic anhydride and fluorophenol (for the difluorinated product) or a mixtures of phenol and fluorophenol (1:4 for the monofluorinated product) in the presence of zinc chloride (10 wt %) catalyst at 170° C. These reaction mixtures were separated by the same methods as the fluorination products and compared by analytical HPLC and spectroscopic measurements. Other fluorinated products may also be available using Fridel-Crafts chemistry.

### Example 2

### Initial Results of Labeling Phenol Red

Separation of the reaction mixture resulting from the fluorination of Phenol Red by HPLC showed the presence of non-reacted PR (about 10%) and three major radioactive products, identical to attributed to mono- (MFPR), di- (DFPR), and tri- (TFPR) fluorophenolsulfonphthaleins (e.g., MFPR corresponds to Mono-Fluoro-Phenol Red). The corrected radiochemical yield of these products varied for different experiments in the range of 3-5%, 6-8%, and 1%, respectively. All the compounds acted as pH indicators with pKₐ values 7.3, 6.4, and 5.9 and absorption of basic forms at 564 nm, 570 nm, and 572 nm for the mono-, di-, and trifluoro derivatives, respectively. The reaction mixture also contained a small amounts of highly absorbing radioactive product with higher retention time, believed to correspond to tetrafluorinated phenolsulfonphthalein.

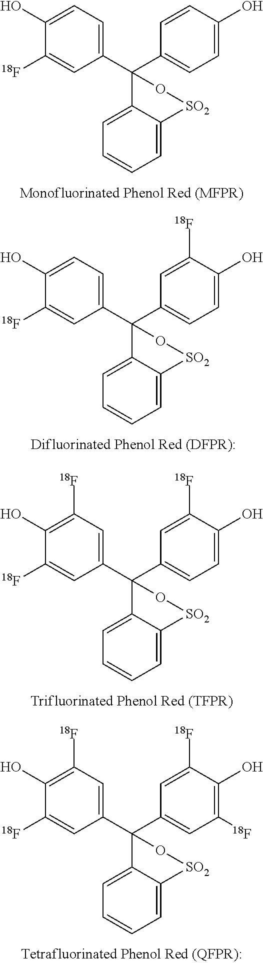 [SMILES: O=S1(=O)OC(c2ccc(O)cc2)(c2ccc(O)c([18F])c2)c2ccccc21 ; O=S1(=O)OC(c2ccc(O)c([18F])c2)(c2ccc(O)c([18F])c2)c2ccccc21 ; O=S1(=O)OC(c2ccc(O)c([18F])c2)(c2cc([18F])c(O)c([18F])c2)c2ccccc21 ; O=S1(=O)OC(c2cc([18F])c(O)c([18F])c2)(c2cc([18F])c(O)c([18F])c2)c2ccccc21]

The presence and identify of non-reacted precursor and three main fluorinated compounds were achieved by comparison with the products of direct fluorination of Phenol Red by HPLC analysis. Two products of the reaction were found to be identical with synthetic monofluorophenol red (MFPR) and difluorophenol red (DFPR), the third product is presumed to be trifluorophenol red (TFPR). The overall fluorination reaction is described in Scheme 1.

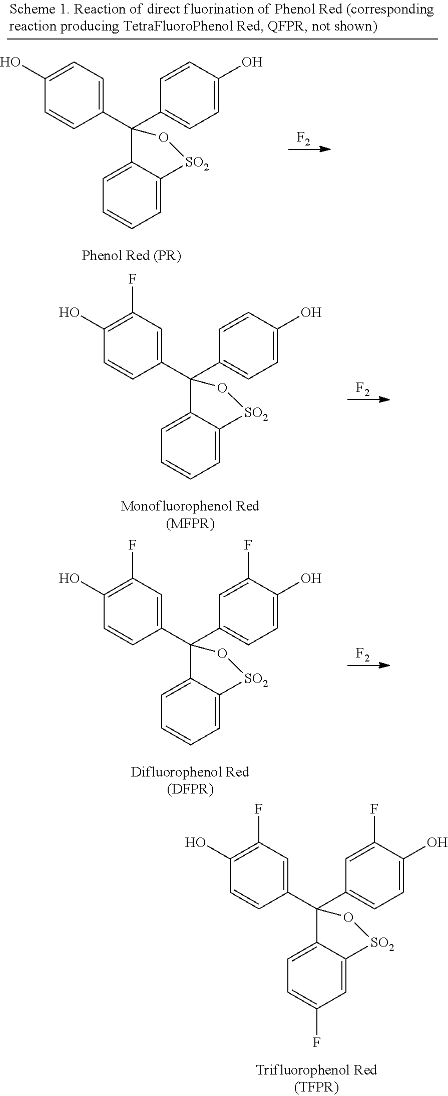

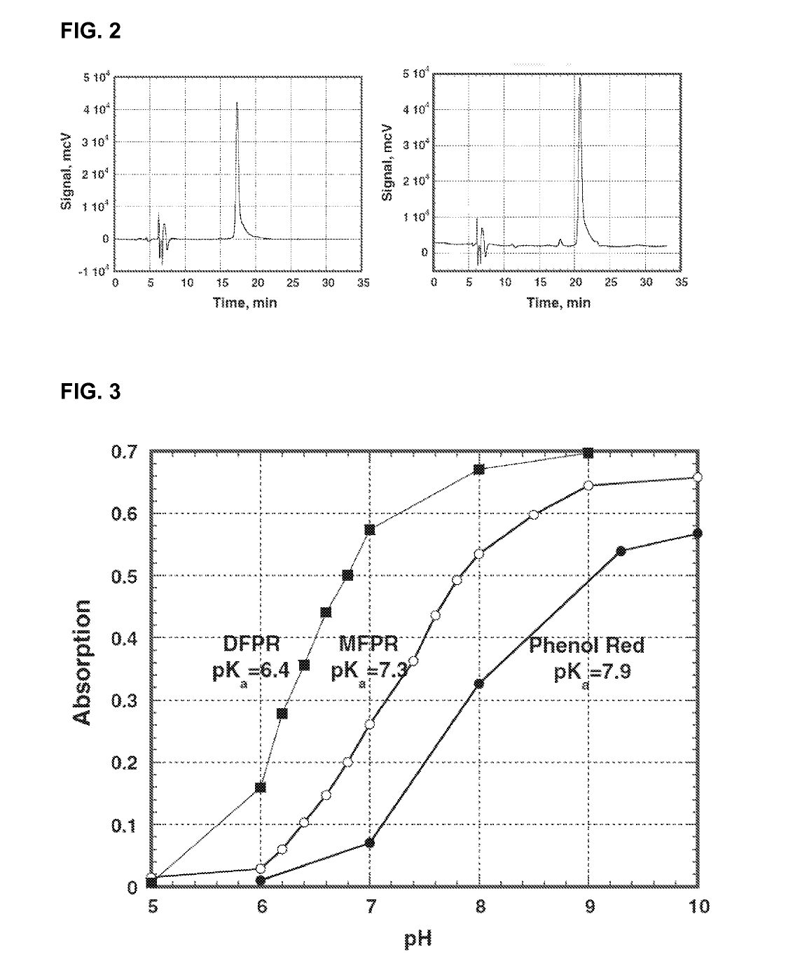

MFPR and DFPR were characterized by comparison of their HPLC signals (FIG. 2) and absorption spectra at different pH (FIG. 3) with subsequent parameters of the same compounds synthesized by the condensation reaction. Behavior of the products was identical in both cases, confirming the identical structure of the compounds.

Quantitative analysis of the reaction products at stoichiometric ratio of fluorine and Phenol Red shows that the mixture contains 6% of non-reacted precursor, 8% of MFPR, 11% of DFPR, and 4% of TFPR. Values of elution time for these compounds on reverse phase HPLC column were consecutively increasing, which correlates with increased hydrophobic character of the molecule after addition of fluorine atom.

Increasing of relative amount of fluorine resulted in simultaneous decreasing of the Phenol Red signal and the yield of reaction products to only few percents. Double excess of fluorine (ratio F₂:PR 2:1) resulted in discoloring of the reaction mixture and presence of only trace amount of the identifiable products.

One of the features indicating that the aromatic system of Phenol Red survives the fluorination is retaining of its indicator properties. Phenol Red is a widely used acid-base indicator with wavelength change from 433 nm to 558 nm at pKₐ=7.9. All the mentioned above products of fluorination also act as pH indicators with increased wavelength and intensity of absorption upon transfer from acidic to basic form FIG. 3 illustrates dependence of intensity of absorption maxima on pH of solution for the basic forms of phenol red and the products of its fluorination. MFPR act like an indicator with absorption maximum at 436 nm for acidic and 564 nm for basic form. Transition between these two forms occurs at pKₐ=7.3. Basic form of DFPR has an absorption maxima at 428 nm and converts into basic form with 570 nm absorption at pKₐ=6.4. Trifluorinated derivative also acted as an indicator with absorption maximum for basic form at 572 nm and pKₐ=5.9.

### Example 3

### Results for Cresol Purple

The reaction between diluted fluorine gas and Cresol Purple in acidic solution was found to be similar to recently to the fluorination of the other triaryl precursors. Analytical HPLC of the reaction mixture shows presence of initial compound and three major (and a fourth minor) fluorine-containing products. These compounds demonstrate increasing retention time on reverse-phase column allowing their separation by semi-preparative HPLC similarly to fluorinated derivatives of phenolsulfonphthalein.

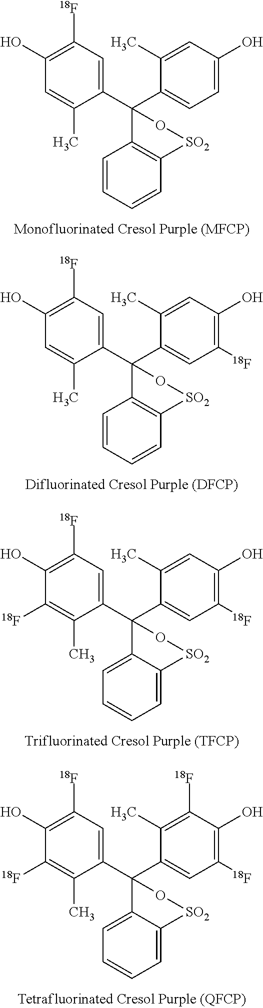 [SMILES: Cc1cc(O)ccc1C1(c2cc([18F])c(O)cc2C)OS(=O)(=O)c2ccccc21 ; Cc1cc(O)c([18F])cc1C1(c2cc([18F])c(O)cc2C)OS(=O)(=O)c2ccccc21 ; Cc1cc(O)c([18F])cc1C1(c2cc([18F])c(O)c([18F])c2C)OS(=O)(=O)c2ccccc21 ; Cc1c(C2(c3cc([18F])c(O)c([18F])c3C)OS(=O)(=O)c3ccccc32)cc([18F])c(O)c1[18F]]

Note here that the position of the fluorine in the monofluorinated Cresol Purple is shown as in the position ortho to the hydroxy group and para to the methyl group, it is possible that another additional isomer exists, wherein the fluorine is added in the position between the hydroxy and methyl groups. Such an additional isomer is suggested, for example, by the two peaks attributed to Cresol Purple in FIG. 1. Such structure (and the di- and tri-fluoro analogs) are considered within the scope of the present invention.

Mass-spectrometric investigation of the fractions containing three fluorination products showed main peaks at m/z 399.070, 417.061, and 435.051 for negative mode and m/z 401.090, 419.081, and 437.071 for positive mode. These numbers correspond to calculated values for deprotonated and protonated forms of mono-, di-, and trifluorinated derivatives of Cresol Purple, abbreviated as MFCP, DFCP, and TFCP in Scheme 2. Semi-preparative HPLC also yielded a UV absorbing peak with retention time higher than TFCP. It was not possible to collect enough of this compound for optical and NMR analysis, but mass spectra contained distinct signals at 453.042 for negative mode and 455.061 amu for positive mode, suggesting synthesis of tetrafluorinated derivative of Cresol Purple.

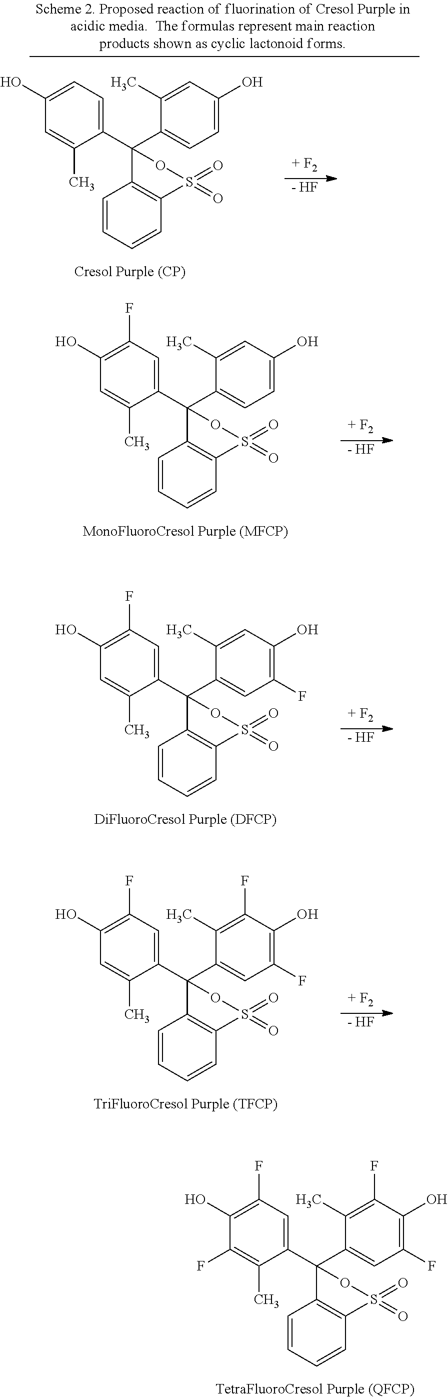

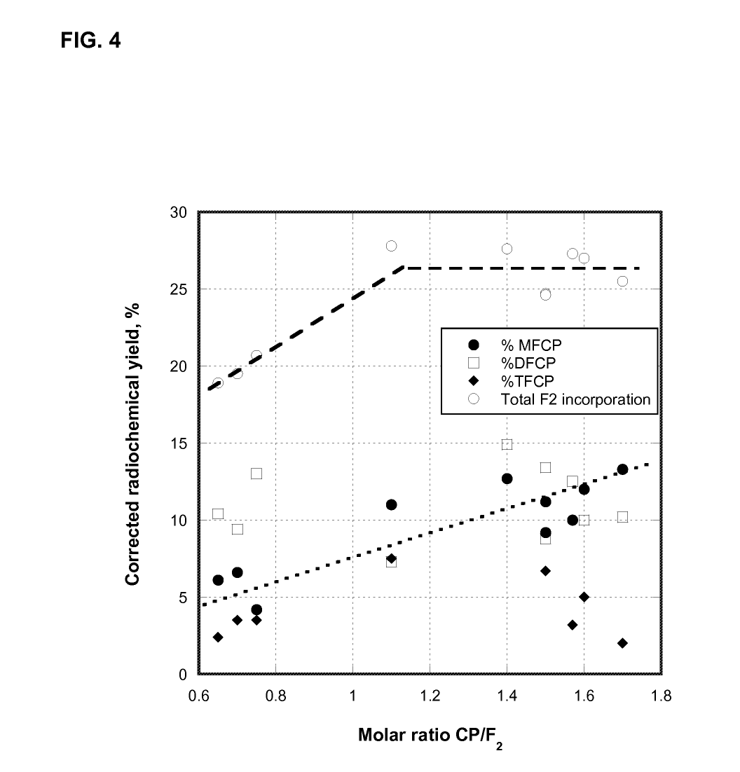

Yields of the reaction products were variable and depended on the reagents ratio. Equimolar quantities of reagents resulted in production 9-10% of MFCP, 5-6% of DFCP, and 1-2% of TFCP. Excess of Cresol Purple over F₂ apparently increased efficacy of fluorine incorporation, yielding 13% of MFCP, 10% of DFCP, and 1% of TFCP (vs amount of fluorine) at two-fold Cresol Purple excess. Excess of fluorine increased fraction of more fluorinated products, but their overall yields drop due to destruction of the molecules. For example, 50% excess of fluorine causes production of 4% of MFCP, 6.5% of DFCP, and 2.5% of TFCP. More than a two-fold excess of fluorine over Cresol Purple caused destruction of the most of the reaction products. In all the cases about 10% of the initial compound remained in the reaction mixture. In order to optimize the conditions for the synthesis of the different fluorinated derivatives of Cresol Purple, the fluorination reaction was done with varying amounts of fluorine and precursor. Dependence of corrected radiochemical yields of the fluorinated products on the ratio of the precursor to fluorine is shown on FIG. 4. As shown in FIG. 4, increasing the amount of the precursor resulted in increasing of the yield of monofluorinated product (closed circle, dotted line). An excess of fluorine—low cresol purple (CP)/F₂ ratio—was expected to increase the yields of difluorinated (squares) and trifluorinated (diamonds) products. However, this did not occur, possibly because excess of fluorine destroyed the reaction products, which was evident from decreasing total F₂ incorporation into the fluorinated derivatives (open circles, broken line) at fluorine excess. An excess of precursor caused production of higher amount of monofluorinated derivative, while the main radioactive product at fluorine excess is difluorinated cresol purple. Still, the difference between the yields of the products was quite small and therefore it was reasonable to calculate average radiochemical yields of the products as 9.6±1% for MFCP, 11±0.7% for DFCP, and 4.2±0.7% for TFCP

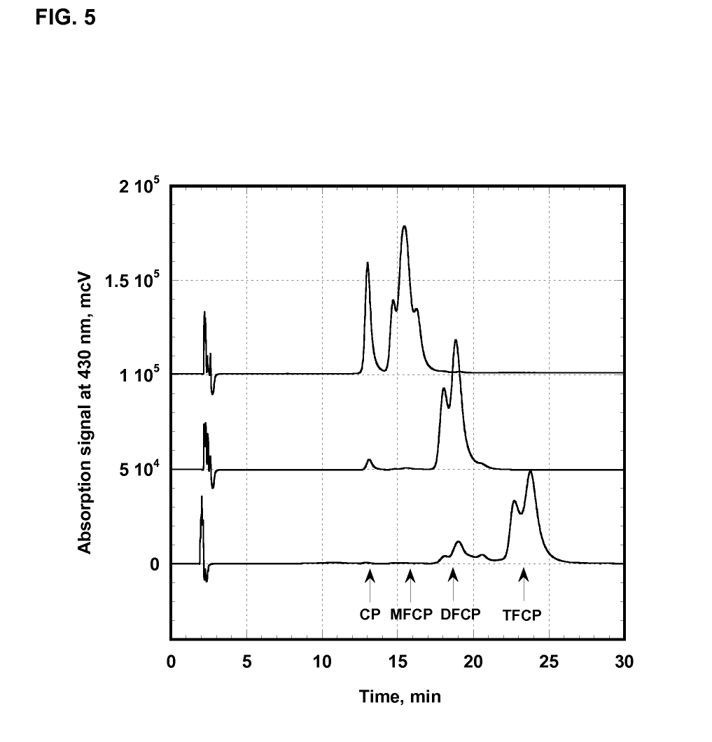

Analytical HPLC of the reaction products revealed an unusual feature for the fluorinated derivatives of cresolsulfonphthalein. For every preparation their HPLC signals were splitting into very close peaks (FIG. 5). This effect did not depend on solvent and pH of eluting buffer, suggesting presence of different compounds with similar properties rather than existence of acid-base or tautomeric equilibrium in solution. To identify these products, correspondent fractions from analytical HPLC were collected and analyzed them by mass-spectroscopy. The mass-spectra were found to be identical for all the peaks from each group, suggesting existence of the structural isomers for each fluorinated compound.

Comparisons of ¹H-NMR spectra of cresolsulfonphthalein and its fluorinated derivatives indicated positions of the molecule fluorination. The NMR spectrum of Cresol Purple has three groups of peaks: a sharp signal from two methyl groups at δ 1.933 (s, 6H); three complex multiplets from sulfonated ring: δ 7.10 (1H), δ 7.70 (2H), and δ 8.03 (1H); and three signals from phenol rings: δ 6.47 (dd, J=2.2, 8.6 Hz, 2H), δ 6.66 (t, J=8.6 Hz, 2H), and δ 6.61 (2H). The later signal overlaps with a right peak of the triplet, which masks its split (presumably into doublet with J=2.2 Hz). In the spectrum of DFCP the first two groups of peaks were slightly shifted, but the number of the peaks and integral intensity of the protons were not altered. The signal from methyl groups is represented as a broad peak between 1.9 and 2.2 ppm (6H), and the signals from sulfonated ring retain the same shape with positions shifted to δ 7.02 (1H), δ 7.60 (2H), and δ 7.80 (1H). These results suggested that first two fluorine atoms in fluorinated derivatives of Cresol Purple do not substitute hydrogen atoms in methyl group or the sulfonated ring. On the contrary, the spectrum of DFCP in the range of phenol rings (δ 6.3-6.8) is significantly changed. Integration of these signals clearly showed existence of four protons of this type, represented as several hardly interpreted sharp and broad peaks. These results indicated that first two fluorine atoms in fluorinated derivatives of Cresol Purple do not substitute hydrogen atoms in methyl group or the sulfonated ring, suggesting fluorination of the molecule into the phenol rings. The complicated NMR spectrum in this range can be associated with signal splitting and overlapping due to existence of structural isomers of DFCP, which were visible in analytical HPLC chromatograms. The presence of these isomers also explains the difficulty in obtaining clear ¹⁹F spectra of these compounds. The spectrum of TFCP shows similar to DFCP signals of methyl groups (6H between 1.8 and 2.1 ppm) and sulfonated ring (δ 7.06 (1H), δ 7.62 (2H), and δ 7.81 (1H)) with the same difference in the range phenol rings. It has several hardly interpreted signals with total integrated intensity from three protons, suggesting addition of the third fluorine atom into the phenol rings.

The precise position of the fluorine atoms could not be directly confirmed from ¹⁹F NMR data, but it can be assumed from the general considerations. Central carbon atom at C¹

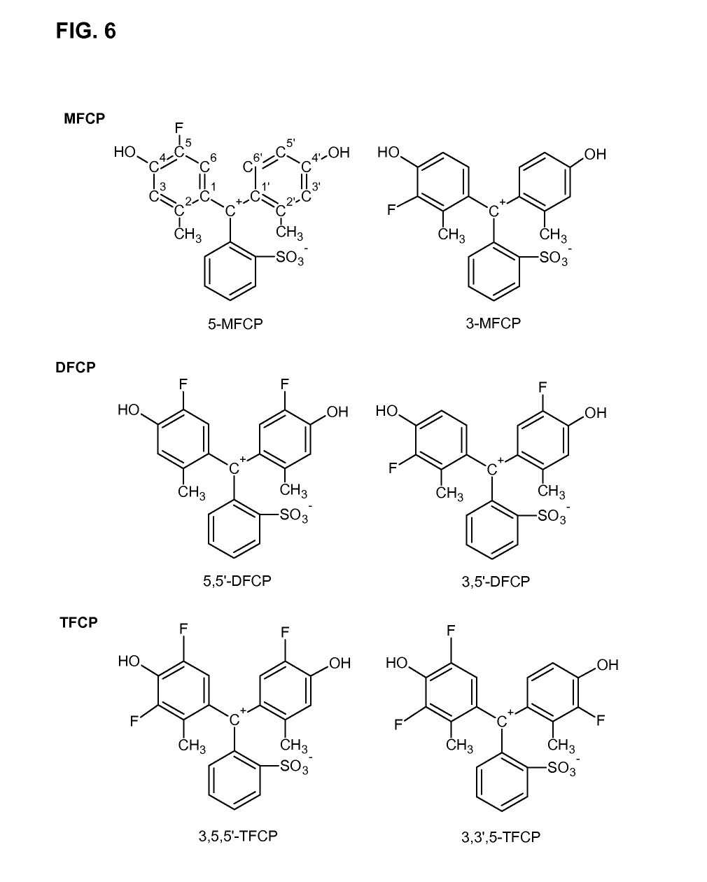

(FIG. 6) has a partial positive charge and promotes meta-orientation into positions C³ and C⁵, deactivating C⁶ atom. Other substitute groups 2-methyl and 4-hydroxyl are ortho-/para-directing groups. All the three groups are located in coordinated positions, promoting substitution of hydrogen into 3^{rd} or 5^{th} position and prohibiting fluorine incorporation into 6^{th} position.

Substitution by fluorine atom at 3^{rd} position seems to be less preferable due to steric hindering by adjacent methyl and hydroxyl groups. Another potential restriction against fluorination of the molecule at C³ may appear, if the reaction mechanism involves intermediate generation of acetyl hypofluorite. Reaction of aromatic ring with this by-product requires availability of two adjacent non-substituted carbon atoms, which is possible only for C⁵-C⁶ couple and will result in fluorine incorporation strictly into 5^{th} position. See Scheme 2. However, presence of isomers of fluorinated cresolsulfonphthalein, as well as the fact of existence of the trifluorinated derivative with all three fluorine atoms in the phenol rings, shows a possibility of the molecule fluorination into both C³ and C⁵ positions.

Availability of both fluorination positions results in appearance of different isomers of fluorinated cresolsulfonphthaleins. Existence of these isomers explains complicated character of NMR spectra of the compounds. The isomers are slightly separable only by analytical HPLC, but preparation of their large quantities in this way is much harder and rather impractical. The indicator properties of the isomers (both pKₐ and absorption wavelengths) should be very close, allowing their application as pH indicators without further separation. The structures of the main isomers of Cresol Purple are presented in FIG. 6. DFCP could have two other isomers (3,5-DFCP and 3,3′-DFCP). A small peak between major signals of DFCP and TFCP (located at 20-21 min in two lower curves on FIG. 5) was collected and analyzed by mass-spectroscopy. It was found to have the same mass as DFCP and most likely represents one of these minor isomers.

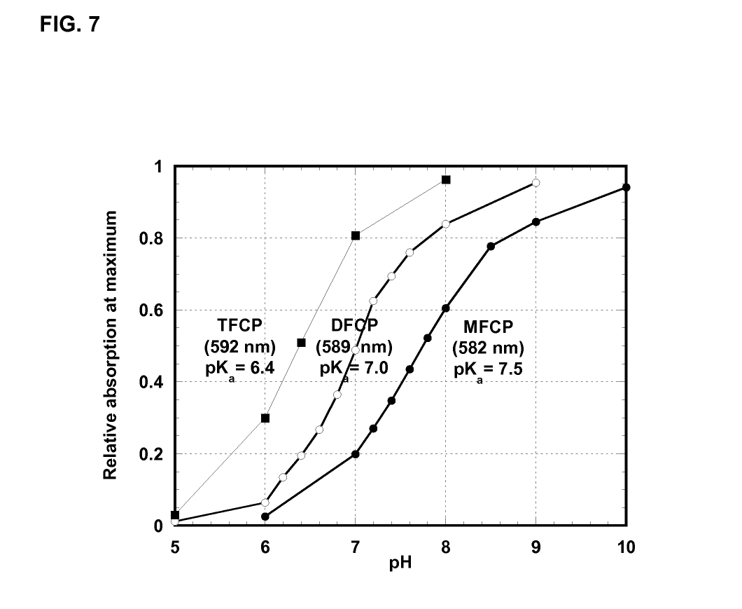

Introduction of fluorine atom(s) into molecules of Cresol Purple caused alteration of pH indicator properties in water solution. The pKₐ values of the fluorinated derivatives of CP were determined from light absorption of the purified fractions, containing individual compounds. The intensity of absorption of the basic forms were studied, because their extinction coefficients were higher and their absorbance maxima were located in more convenient area of the spectrum. The measurements were taken at absorption maxima and were found to be at 582 nm, 589 nm, and 592 nm for MFCP, DFCP, and TFCP, respectively. The results indicated that introduction of fluorine atom into the molecule of Cresol Purple caused a decrease in the pKₐ of the indicator from 8.3 for CP to 7.5 for MFCP, 7.0 for DFCP, and 6.4 for TFCP (FIG. 7).

### Example 4

### Results of Labeling Cresol Red and Thymol Blue

Fluorination of ortho-cresolsulfonphthalein (Cresol Red, CR) yielded only two radioactive products. Mass-spectroscopic investigation resulted in molecular masses equal to 399.070 and 417.061 amu for negative mode, and 401.090 and 419.081 amu for positive mode, suggesting synthesis of MonoFluoroCresol Red (MFCR) and DiFluoroCresol Red (DFCR) derivatives. Radiochemical yields of these two products were 4-6% and 10-12% respectively. The compounds act like pH indicators with pKₐ at 7.6 and 6.8, and absorption maxima of basic forms at 576 nm and 582 nm, for the mono- and difluoro-derivatives, respectively (this compares with pKa of the non-fluorinated Cresol Red of 8.2 and absorption maxima of basic form at 572 nm).

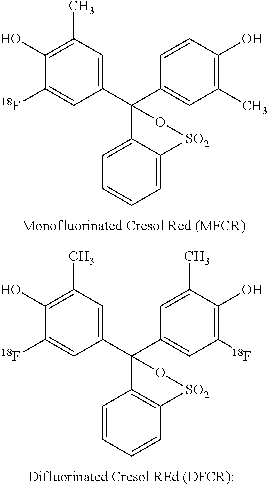 [SMILES: Cc1cc(C2(c3cc(C)c(O)c([18F])c3)OS(=O)(=O)c3ccccc32)ccc1O ; Cc1cc(C2(c3cc(C)c(O)c([18F])c3)OS(=O)(=O)c3ccccc32)cc([18F])c1O]

Using the same experimental protocols, it was also possible to fluorinate another compound of this class, thymolsulfonphthalein, usually called Thymol Blue (TB). Fluorination of this compound yielded two major radioactive products with molecular masses 483.17 and 501.16 amu for negative mode, which corresponds to MonoFluoro Thymol Blue (MFTB) and DiFluoro Thymol Blue (DFTB) derivatives. Radiochemical yield of the products was 10% for MFTB and 6.5% for DFTB. Incorporation of fluorine atoms into the molecule of thymol blue caused a shift of the pKₐ and absorption similarly to other derivatives of phenolsulfonphthalein. For Thymol Blue, the pKₐ was found to be 8.85, with absorption of basic form at 597 nm. For the monofluorinated derivative, the pKₐ was found to be 8.25, while shift of absorption of basic form was quite small (to 599 nm). The second fluorine atom in the molecule caused more significant alteration of its properties, giving a pKₐ value of 7.65 and the absorption maximum at 605 nm.

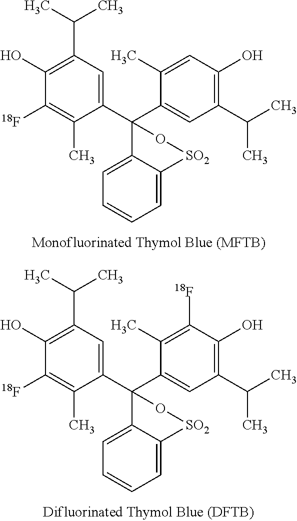 [SMILES: Cc1cc(O)c(C(C)C)cc1C1(c2cc(C(C)C)c(O)c([18F])c2C)OS(=O)(=O)c2ccccc21 ; Cc1c(C2(c3cc(C(C)C)c(O)c([18F])c3C)OS(=O)(=O)c3ccccc32)cc(C(C)C)c(O)c1[18F]]

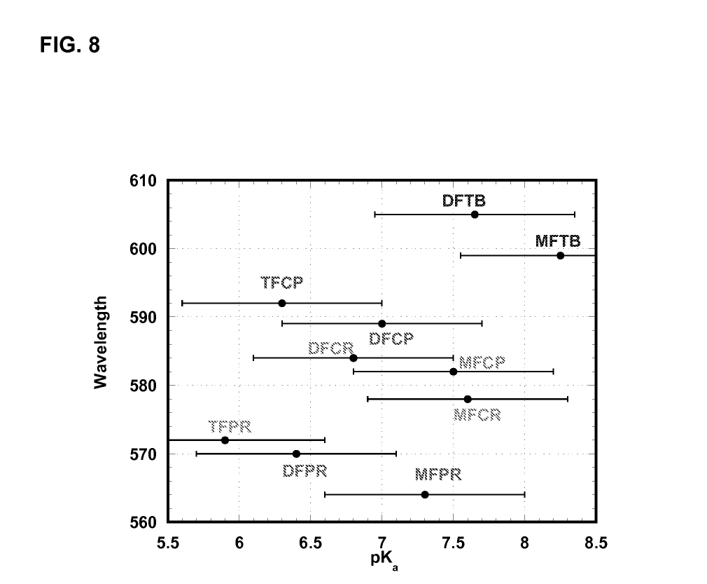

A summary of data on the spectroscopic and the acid-base properties of the fluorinated derivatives of phenolsulfonphthalein and its analogs is presented in FIG. 8.

### Example 5

### Results of Labeling Phenolphthalein and Naphtholphthalein

Phenolphthalein and naphtholphthalein were each fluorinated using a modification of the General Procedure of Example 1. While it is possible to fluorinate Phenol Red and its sulfonated cogeners/analogs in glacial acetic acid (as well as the more acidic fluoro/perfluoro C₁₋₃ carboxy acids), phenolphthalein and naphtholphthalein required that the solvent comprise an acid stronger than acetic acid (e.g., trifluoroacetic acid). Using trifluoroacetic acid as the solvent in a procedure otherwise as described in the General Procedure cited above, it was possible to isolate and identify at least mono- and difluoro derivatives of each phenolphthalein and naphtholphthalein. Other tri- and tetrafluoro derivatives may also be available.

All of the fluorinated compounds acted like pH indicators, with mono- and difluorophenolphthalein exhibiting a pKₐ at 9.3 and 8.7 and absorption maxima of basic forms at 558 nm and 564 nm, respectively (compared with a pKa of the non-fluorinated phenolphthalein of 9.75 and absorption maxima of basic form at 553 nm) and mono- and difluoronaphtholphthalein exhibiting a pKₐ at 7.7 and 7.2, and absorption maxima of basic forms at 660 nm and 668 nm, respectively (compared with a pKa of the non-fluorinated naphtholphthalein of 7.95 and absorption maxima of basic form at 653 nm).

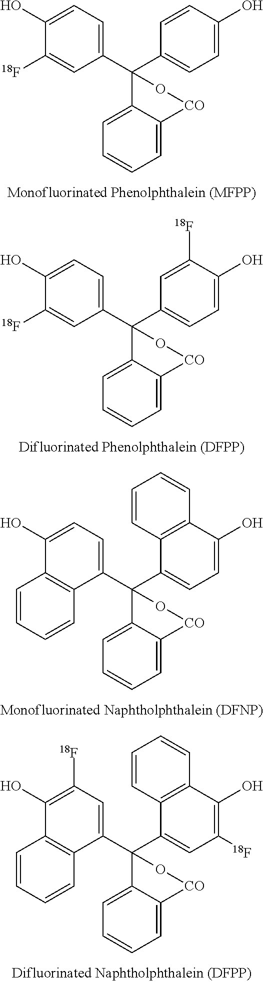 [SMILES: O=C1OC(c2ccc(O)cc2)(c2ccc(O)c([18F])c2)c2ccccc21 ; O=C1OC(c2ccc(O)c([18F])c2)(c2ccc(O)c([18F])c2)c2ccccc21 ; O=C1OC(c2ccc(O)c3ccccc23)(c2ccc(O)c3ccccc23)c2ccccc21 ; O=C1OC(c2cc([18F])c(O)c3ccccc23)(c2cc([18F])c(O)c3ccccc23)c2ccccc21]

### Example 6

### Additional Comments

Comparisons of the fluorination results for different indicators are consistent with the generally identified positions of incorporation of fluorine atoms described herein. Investigation of the fluorination of Cresol Purple have shown incorporation of four fluorine atoms, and the third atom was clearly located in the phenol ring in ortho position to OH-group. The present results show that Cresol Red and Thymol Blue are able to attach only two fluorine atoms. Both of these molecules have two ortho positions occupied by aliphatic groups (methyl and isopropyl, respectively), which prevents addition of two more fluorine atoms into these positions. This is consistent with a conclusion that fluorination of phenolsulfonphthalein derivatives occurs only into ortho positions of the phenol ring; that is, the sulfonic group (at least for the sulfonated derivatives) completely deactivates aromatic ring for this kind of substitution.

It is also notable that the precursor indicators described herein underwent successful fluorination only while dissolved in glacial acetic (or stronger carboxylic) acid, and consistent with the resonance structures described herein. While the acidic forms of these indicator precursors are poorly soluble in such solvents, salts of these precursors (including both monosodium and disodium forms) can be dissolved in such solvents, where they are immediately converted into the acid form. This form ultimately precipitates from the solution, but this process occurs quite slowly, allowing several minutes for conducting of the fluorination reaction. Because of this, the reaction has to be performed immediately with freshly prepared solution of the salts (e.g., sodium salt) in the carboxylic acid (e.g., acetic acid), and the product contains a significant (up to 10%) amount of the precursor even at excess of fluorine. These peculiar conditions can be an explanation of the fact that the reaction of direct fluorination of phenolsulfonphthalein and its derivatives was not observed previously.

The radiochemical yields of all of the compounds discussed in these Examples varied from 5 to 10% depending on the precursor and the number of fluorine atoms. Increasing the relative amount of fluorine did not shift the reaction to higher overall yield of the fluorinated products, but causes their destruction. This effect could be associated with aggressive nature of fluorine gas and the destruction of the organic moieties. It was not possible to observed higher yields of fluorinated products under these conditions. Still their yields are high enough to prepare amounts of the compounds in quantities that would be useful even for human objects. After a normal 2 hour deuteron irradiation on IBA Cyclone 18/9 at 20 μA/minute beam current we are able to synthesize 10-15 mCi of the labeled pH indicators, which is comparable to standard FDG patient dose (15 mCi). This kind of reactivity perhaps disfavored bulk preparation of the reaction products, but provides for a convenient processes on micro scale level, for example the labeling of the molecule with ¹⁸F. Thus far, electrophilic fluorination with [¹⁸F]—F₂ gas has been performed for milligram quantities of reagents, usually producing a complex mixture of products with similar structure. Again, thus far, separation of this kind of mixture required application of semi-preparative HPLC, allowing for the isolation and capture of all the products from the mixture. It was not yet possible to perform a proper separation of mono-, di-, and trifluorinated phenol red derivatives using semi-preparative hydroxylated C-18 column from Phenomenex. As it is evident from FIG. 2, products of fluorination were essentially pure, which validated a separation method and allowed their further application as pH indicators.

As those skilled in the art will appreciate, numerous modifications and variations of the present invention are possible in light of these teachings, and all such are contemplated hereby. For example, in addition to the embodiments described herein, the present invention contemplates and claims those inventions resulting from the combination of features of the invention cited herein and those of the cited prior art references which complement the features of the present invention. Similarly, it will be appreciated that any described material, feature, or article may be used in combination with any other material, feature, or article, and such combinations are considered within the scope of this invention.

The disclosures of each patent, patent application, and publication cited or described in this document are hereby incorporated herein by reference, each in its entirety.

1. A compound having a structure of Formula I, II, III, or IV, or a salt thereof: 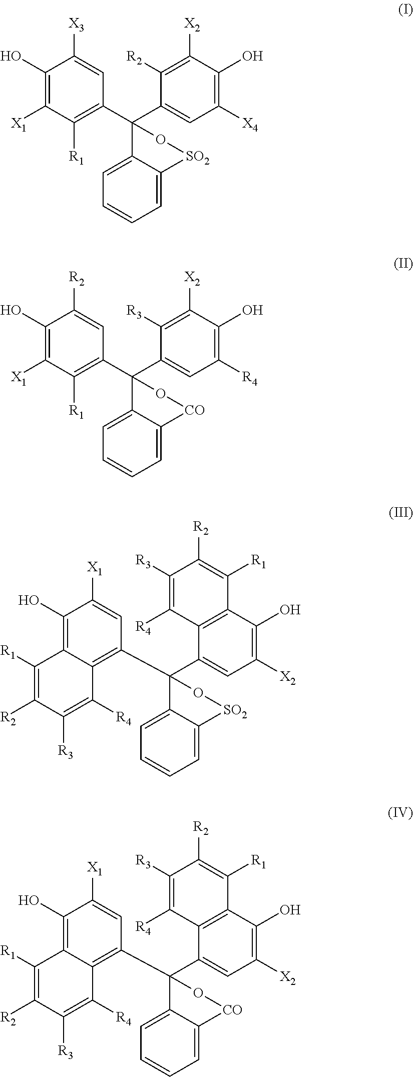
wherein
X₁ and X₂ are independently, at each occurrence, hydrogen or fluorine;
R₁, R₂, R₃, and R₄, are independently, at each occurrence, hydrogen, optionally substituted C₁₋₆ alkyl; optionally substituted C₃₋₆ cycloalkyl, optionally substituted C₂₋₆ alkenyl; optionally substituted alkoxy, optionally substituted aryl, optionally substituted heteroalkyl, optionally substituted heteroaryl, or halo;
X₃ and X₄ are independently, at each occurrence, hydrogen, fluorine, bromine, chlorine, iodine, optionally substituted C₁₋₆ alkyl; optionally substituted C₃₋₆ cycloalkyl, optionally substituted C₂₋₆ alkenyl; optionally substituted alkoxy, optionally substituted aryl, optionally substituted heteroalkyl, or optionally substituted heteroaryl;
provided at least one of X₁, X₂, X₃, and X₄ is fluorine; wherein
where present, the fluorine is either present at its natural abundance of isotopes at each occurrence or is statistically enriched in the radioisotope ¹⁸F above its natural isotopic abundance relative to the ¹⁹F isotope at each occurrence.

2. The compound of claim 1, wherein R₁, R₂, R₃, R₄, X₃ and X₄ are independently, at each occurrence, hydrogen or linear or branched C₁₋₃ alkyl, preferably methyl.

3. The compound of claim 1, wherein either (a) X₁=fluorine, X₂=X₃=X₄=H; or (b) X₁=X₂=fluorine, X₃=X₄=H; or (c) X₁=X₂=X₃=fluorine, X₄=H, or (d) X₁=X₂=X₃=X₄=fluorine.

4. The compound of claim 1, having a structure: 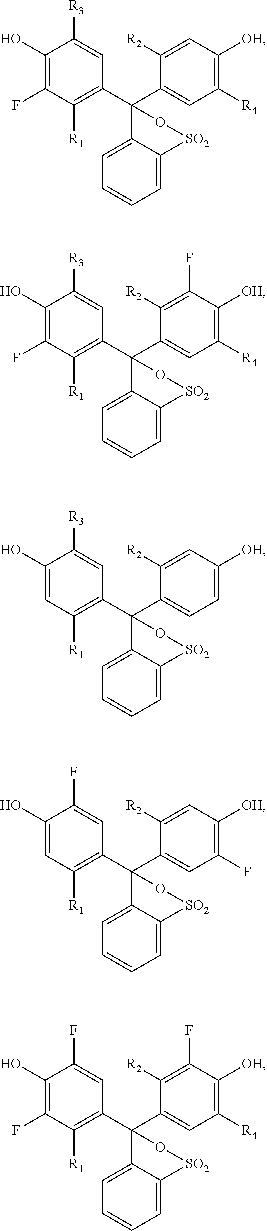 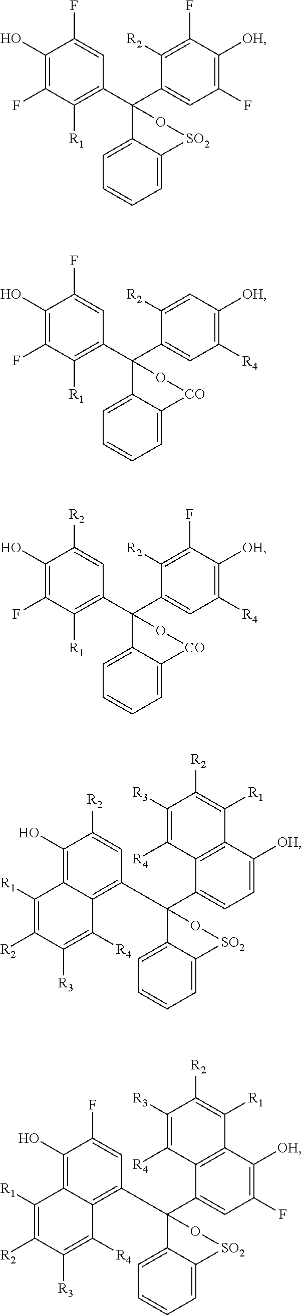 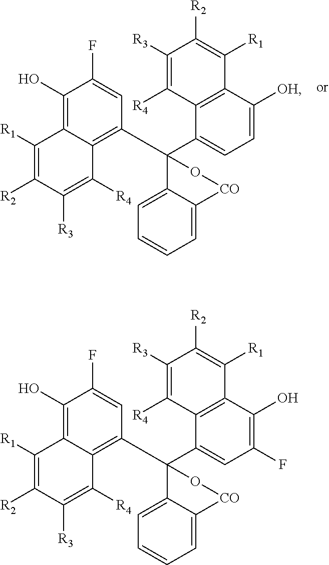
where F represents fluorine being present either statistically at its natural abundance of isotopes at each occurrence or statistically enriched in the radioisotope ¹⁸F above its natural isotopic abundance relative to the ¹⁹F isotope at each occurrence.

5. The compound of claim 1, having a structure: 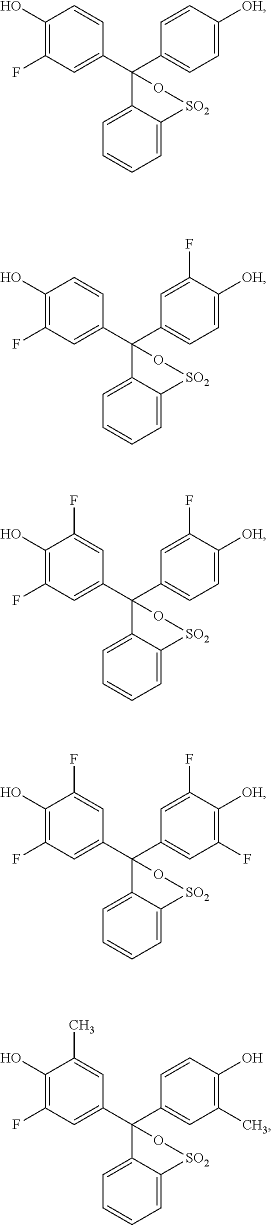 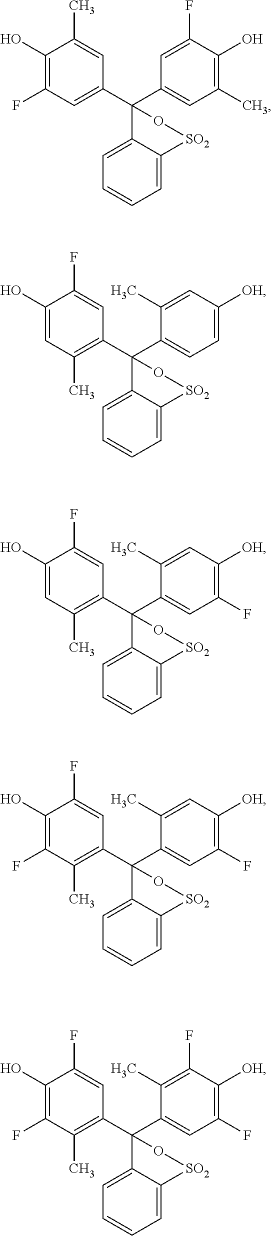 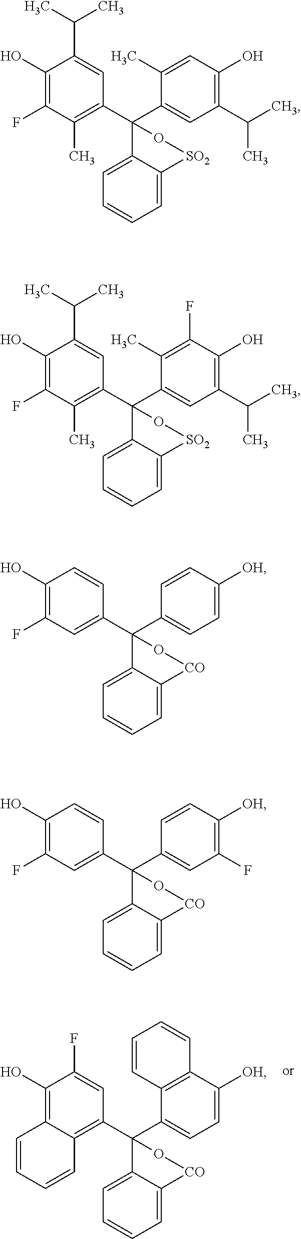 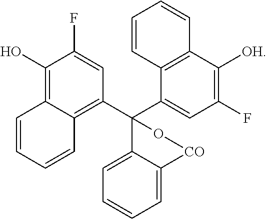
where F represents fluorine being present either statistically at its natural abundance of isotopes at each occurrence or statistically enriched in the radioisotope ¹⁸F above its natural isotopic abundance relative to the ¹⁹F isotope at each occurrence.

6. The compound of claim 1, wherein the fluorine is statistically enriched in the radioisotope ¹⁸F above its natural isotopic abundance relative to the ¹⁹F isotope at each occurrence.

7. A compound prepared by the electrophilic fluorination of a precursor of Formula V, VI, VII, or VIII or a salt thereof: 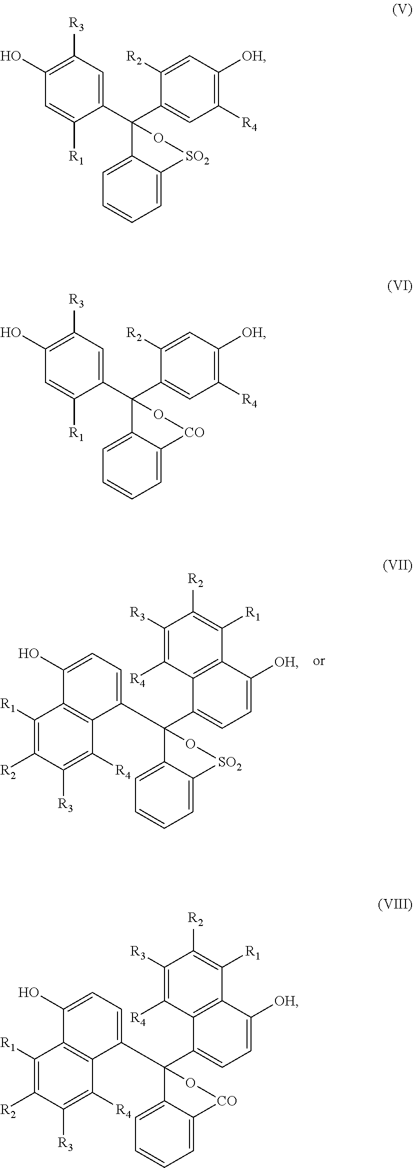
wherein
R₁, R₂, R₃, and R₄ are independently, at each occurrence, hydrogen, optionally substituted C₁₋₆ alkyl; optionally substituted C₃₋₆ cycloalkyl, optionally substituted C₂₋₆ alkenyl; optionally substituted alkoxy, optionally substituted aryl, optionally substituted heteroalkyl, optionally substituted heteroaryl, or halogen;
the electrophilic fluorination reaction comprising reacting the precursor with fluorine gas in a solvent such that the compound contains at least one fluorine atom per compound molecule; wherein either:
(a) the fluorine gas and the at least one fluorine atom per compound molecule consist of fluorine atoms statistically present at their natural isotopic abundance; or
(b) the fluorine gas is radiolabeled to be enriched in ¹⁸F above its natural isotopic abundance relative to the ¹⁹F isotope and the at least one fluorine atom per compound molecule is enriched in ¹⁸F above its natural isotopic abundance relative to the ¹⁹F isotope.

8. The compound of claim 7, wherein R₁, R₂, R₃, R₄, X₃ and X₄ are independently, at each occurrence, hydrogen or linear or branched C₁₋₃ alkyl, preferably methyl.

9. The compound of claim 7, wherein the compound contains one, two, three or four fluorine atoms per compound molecule.

10. A compound prepared by the electrophilic fluorination of a triarylmethane dye precursor, said precursor comprising phenol red, cresol red, cresol purple, thymol blue, bromophenol red, naphthol blue, phenolphthalein, cresolphthalein, thymolphthalein, naphtholphthalein, gentian violet (methyl violet 10B), methyl violet 2B, methyl violet 6B, leucomalachite green (malachite green), brilliant green, pararosaniline, fuchsines, or a salt thereof;
the electrophilic fluorination reaction comprising reacting the precursor with fluorine gas in a solvent such that the compound contains at least one fluorine atom per compound molecule; wherein either:
(a) the fluorine gas and the at least one fluorine atom per compound molecule consist of fluorine atoms statistically present at their natural isotopic abundance; or
(b) the fluorine gas is radiolabeled to be enriched in ¹⁸F above its natural isotopic abundance relative to the ¹⁹F isotope and the at least one fluorine atom per compound molecule is enriched in ¹⁸F above its natural isotopic abundance relative to the ¹⁹F isotope.

11. The compound of claim 10, wherein the precursor comprises phenol red, cresol red, cresol purple, thymol blue, phenolphthalein, or naphtholphthalein.

12. The compound of claim 7, wherein the fluorine gas is radiolabeled to be enriched in ¹⁸F above its natural isotopic abundance relative to the ¹⁹F isotope and the at least one fluorine atom per compound molecule is enriched in ¹⁸F above its natural isotopic abundance relative to the ¹⁹F isotope.

13. The compound of claim 7, wherein the solvent is an organic acid.

14. The compound of claim 13 wherein the organic acid is CH₃COOH, CH₂FCOOH, CHF₂COOH, CF₃COOH, HCOOH, or a mixture thereof

15. The compound of claim 13, wherein the solvent is a fluorinated or perfluorinated C₃₋₇ organic acid.

16. The compound of claim 7, further comprising separating the products of the reaction by chromatography.

17. The compound of claim 16 wherein the chromatography is HPLC

18. A method of determining the concentration of a compound of claim 6 in a biological object comprising measuring emission of positrons from the biological object using positron emission tomography (PET) or single photon emission computer spectroscopy (SPECT).

19. A method of measuring blood volume, or blood flow through the organ or tissue, or the in vivo pH in a biological object, said method comprising incorporating the compound of claim 6 in said biological object and measuring emission of positron correlatable to a concentration of the compound using positron emission tomography (PET) or single photon emission computer spectroscopy, and a spectral response of the compound using light in the visible or ultraviolet ranges, said spectrum representative of the pH of the biological object.

20. A method of detecting pH changes associated with hypoxia or diabetes in a patient, said method comprising:
(a) administering to the patient a compound of claim 6;
(b) optionally allowing the compound to distribute within a biological object (for example blood or a tumor) within the patient;
(c) measuring emission of positrons correlatable to a concentration of the compound using positron emission tomography (PET) or single photon emission computer spectroscopy, and
(d) measuring a spectral response of the compound using light in the visible or ultraviolet ranges, said spectral response being representative of the internal pH of the tissue or organ.

21. A method of preparing a compound of Formula I, II, III, or IV, or a salt thereof, 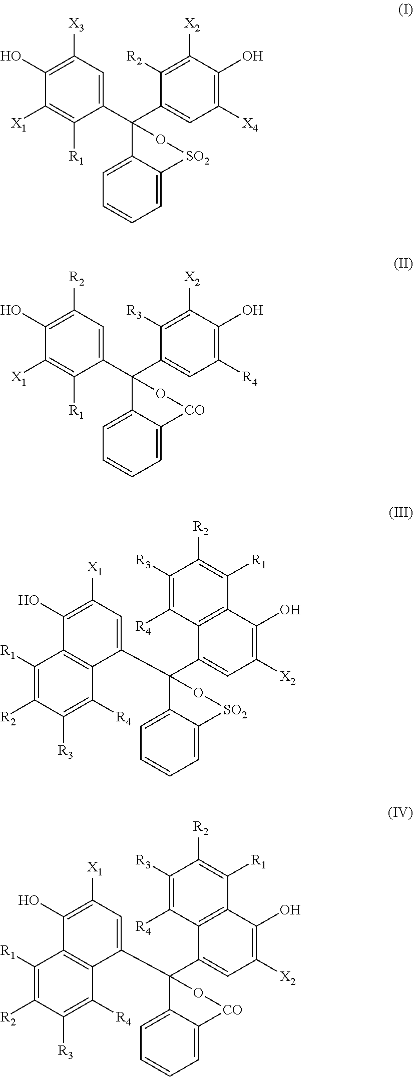
wherein
X₁ and X₂ are independently, at each occurrence, hydrogen or fluorine;
R₁, R₂, R₃, and R₄, are independently, at each occurrence, hydrogen, optionally substituted C₁₋₆ alkyl; optionally substituted C₃₋₆ cycloalkyl, optionally substituted C₂₋₆ alkenyl; optionally substituted alkoxy, optionally substituted aryl, optionally substituted heteroalkyl, optionally substituted heteroaryl, or halo;
X₃ and X₄ are independently, at each occurrence, hydrogen, fluorine, chlorine, bromine, iodine, optionally substituted C₁₋₆ alkyl; optionally substituted C₃₋₆ cycloalkyl, optionally substituted C₂₋₆ alkenyl; optionally substituted alkoxy, optionally substituted aryl, optionally substituted heteroalkyl, or optionally substituted heteroaryl; and
provided at least one of X₁, X₂, X₃, and X₄ is fluorine;
said method comprising reacting a precursor of Formula V, VI, VII, or VIII or a salt thereof, as appropriate, with fluorine gas in a solvent: 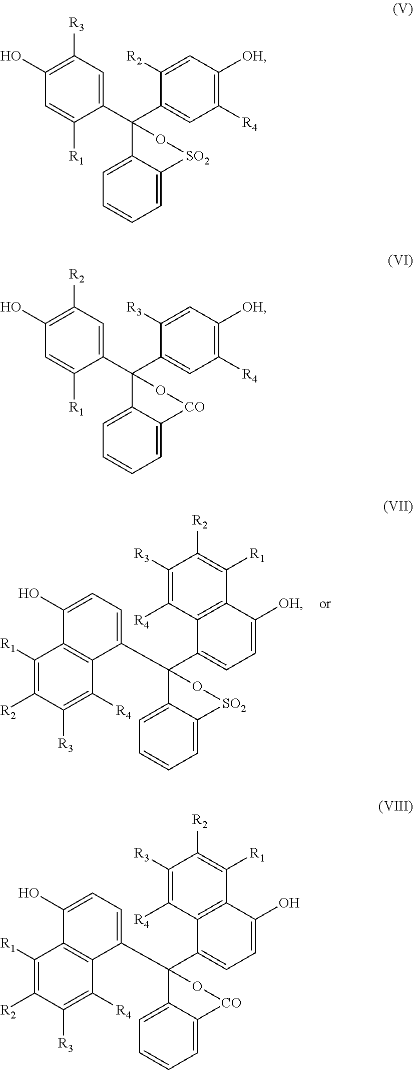
wherein R₁, R₂, R₃, and R₄ have the same meanings as provided for the compounds; and wherein either:
(a) the fluorine gas and at least one fluorine atom per compound molecule is statistically present at their natural isotopic abundance; or
(b) the fluorine gas is radiolabeled to be enriched in ¹⁸F above its natural isotopic abundance relative to the ¹⁹F isotope and the at least one fluorine atom per compound molecule is enriched in ¹⁸F above its natural isotopic abundance relative to the ¹⁹F isotope.

22. The method of claim 21, wherein R₁, R₂, R₃, and R₄ are independently, at each occurrence, hydrogen or linear or branched C₁₋₃ alkyl, preferably methyl.

23. A method of preparing a pH indicator comprising reacting a triarylmethane-based indicator precursor with fluorine gas in a solvent, wherein the triarylmethane-based indicator precursor comprises phenol red, cresol red, cresol purple, thymol blue, bromophenol red, naphthol blue, phenolphthalein, cresolphthalein, thymolphthalein, naphtholphthalein, gentian violet (methyl violet 10B), methyl violet 2B, methyl violet 6B, leucomalachite green (malachite green), brilliant green, pararosaniline, fuchsines, or a salt thereof; wherein either:
(a) the fluorine gas contains fluorine atoms statistically present at their natural isotopic abundance; or
(b) the fluorine gas is radiolabeled to be enriched in ¹⁸F above its natural isotopic abundance relative to the ¹⁹F isotope and the at least one fluorine atom per compound molecule is enriched in ¹⁸F above its natural isotopic abundance relative to the ¹⁹F isotope.

24. The method of claim 23, wherein the triarylmethane-based indicator precursor comprises phenol red, cresol red, cresol purple, thymol blue, phenolphthalein, or naphtholphthalein or a salt thereof.

25. The method of claim 21, wherein the fluorine gas is radiolabeled to be enriched in ¹⁸F above its natural isotopic abundance relative to the ¹⁹F isotope and the at least one fluorine atom per compound molecule is enriched in ¹⁸F above its natural isotopic abundance relative to the ¹⁹F isotope

26. The method of claim 21, wherein the solvent is an organic acid.

27. The method of claim 26 wherein the organic acid is CH₃COOH, CH₂FCOOH, CHF₂COOH, CF₃COOH, HCOOH, or a mixture thereof.

28. The method of claim 21, further comprising separating the products of the reaction by chromatography, preferably HPLC.
